(19) United States
(12) Patent Application Publication
(10) Pub. No.: US 2013/0210045 A1
(43) Pub. Date: Aug. 15, 2013

(54) BIOMARKER FOR FATIGUE, AND USE THEREOF

(71) Applicants: Yosky Kataoka, Kobe-shi, Hyogo (JP); Guanghua Jin, Kobe-shi, Hyogo (JP); Yasuyoshi Watanabe, Kobe-shi, Hyogo (JP); Seiki Tajima, Kobe-shi, Hyogo (JP); Tomoyoshi Soga, Tsuruoka-shi, Yamagata (JP); Hirohiko Kuratsune, Toyonaka-shi, Osaka (JP); Emi Yamano, Osaka-shi, Osaka (JP)

(72) Inventors: Yosky Kataoka, Kobe-shi, Hyogo (JP); Guanghua Jin, Kobe-shi, Hyogo (JP); Yasuyoshi Watanabe, Kobe-shi, Hyogo (JP); Seiki Tajima, Kobe-shi, Hyogo (JP); Tomoyoshi Soga, Tsuruoka-shi, Yamagata (JP); Hirohiko Kuratsune, Toyonaka-shi, Osaka (JP); Emi Yamano, Osaka-shi, Osaka (JP)

(73) Assignees: RIKEN, Wako-shi, Saitama (JP); Hirohiko KURATSUNE, Toyonaka-shi, Osaka (JP)

(21) Appl. No.: 13/805,449

(22) PCT Filed: Aug. 22, 2011
(86) PCT No.: PCT/IB2011/001917

§ 371 Date: Mar. 7, 2013

(30) Foreign Application Priority Data
Feb. 21, 2011 (JP) 2011-034875

(51) Int. Cl. C12Q 1/54; C12Q 1/34; C12Q 1/527; C12Q 1/32; C12Q 1/48; C12Q 1/52

(52) U.S. Cl. CPC C12Q 1/54; C12Q 1/485; C12Q 1/52; C12Q 1/527; C12Q 1/32; C12Q 1/34
USPC 435/14; 435/4; 435/15; 435/16; 435/18; 435/26; 435/287.1; 204/451; 204/452; 204/601; 204/603

(57) ABSTRACT

Ratios of measured values of glucose, citrate, and cis-aconitate in a biological sample obtained from a subject to measured values of glucose, citrate, and cis-aconitate in a biological sample obtained from a healthy subject are calculated, and the glucose ratio, the citrate ratio, and the cis-aconitate ratio are used to evaluate and/or assess fatigue. Similarly, an isocitrate ratio, a succinate ratio, a malate ratio, and a lactate ratio are calculated, and these ratios are used together with the three ratios to evaluate and/or assess fatigue. This makes it possible to objectively and easily diagnose and evaluate fatigue.

### TECHNICAL FIELD

The present invention relates to a biomarker for evaluating fatigue and to evaluation and diagnosis of fatigue with the use of the biomarker.

### BACKGROUND ART

According to an epidemiological survey in Japan, approximately 60% of Japanese citizens are in subjective fatigue, and half or more of those in subjective fatigue are suffering from fatigue continuing for 6 month or longer (i.e., chronic fatigue). Moreover, approximately half of those feeling chronic fatigue are complaining about a decrease in work or study efficiency. It is said that such chronic fatigue causes an economic loss of one trillion and two hundred billion yen. Those feeling chronic fatigue include patients of chronic fatigue syndrome whose main symptom is strong feeling of fatigue and whose cause has not been fully figured out yet.

Most of conventional fatigue diagnoses are based on subjective perception of a patient. Therefore, establishment of an indicator for objectively diagnosing fatigue has been desired. Patent Literature 1 discloses a technique focusing on viral infection, which is one of causes for immune depression. In Patent Literature 1, the viral infection is used as an indicator for evaluating a fatigue level. Non-Patent Literature 1 discloses an attempt to objectively measure/evaluate fatigue with the use of acceleration plethysmogram.

### CITATION LIST

Patent Literature 1
Japanese Patent Application Publication, Tokukai, No. 2007-330263 A (Publication Date: Dec. 27, 2007)

Patent Literature 2
Japanese Patent Application Publication, Tokukaihei, No. 9-77688 A (Publication Date: Mar. 25, 1997)

Patent Literature 3
Japanese Patent Application Publication, Tokukaihei, No. 9-59161 A (Publication Date: Mar. 4, 1997)

Non-Patent Literature 1
“The evaluation of fatigue by acceleration plethysmogram”, Koji YAMAGUCHI et al, Journal of Clinical and Experimental Medicine Feature Article “Latest Science of Fatigue—Suggestion from Japan for Anti-fatigue and Anti-overwork”, Vol. 228, No. 6, 646-653 (2009)

Non-Patent Literature 2
“Development of human fatigue biochenmical biomarkers”, Osami KAJIMOTO, Journal of Clinical and Experimental Medicine Feature Article “Latest Science of Fatigue—Suggestion from Japan for Anti-fatigue and Anti-overwork”, Vol. 228, No. 6, 659-663 (2009)

Non-Patent Literature 3
“Mental and physical fatigue-related biochemical alterations”, Nozaki S. et al. Nutrition 25, 51-57 (2009)

### SUMMARY OF INVENTION

### Technical Problem

A living body has by nature a mechanism of maintaining homeostasis, i.e., a mechanism of recovering from disturbed homeostasis. Development of a technique of objectively evaluating fatigue in accordance with such a mechanism intrinsic to a living body has been desired. Specifically, identification of a biomarker that allows biological information to be quantified or to be converted into numerical values on the basis of concentration of a chemical substance contained in a biological sample has been expected.

According to the technique disclosed in Patent Literature 1, a body fluid is taken from a subject, an amount of human herpes virus in the body fluid is measured, and a relationship between the amount of human herpes virus and a fatigue level is evaluated. This technique is, however, for observing behavior of a virus having infected a human (host), and is not for measuring a biomarker reflecting a fatigue mechanism in a human. According to the technique disclosed in Patent Literature 1, it is therefore impossible to evaluate a fatigue status of an individual subject and to provide a treatment or prevention method for fatigue. The technique disclosed in Non-Patent Literature 1 has an advantage of being non-invasive, but requires measurement of finger plethysmogram with the use of a special equipment and requires data processing based on a special principle, and therefore has poor applicability. Non-Patent Literature 2 describes a list of candidates for a biochemical marker for evaluating fatigue with the use of blood, saliva, or urine, and mentions that these factors are deeply relevant to fatigue and feeling of fatigue. Non-Patent Literature 3 mentions that some factors in various biological samples change their amounts in association with mental fatigue and physical fatigue. However, many of the factors described in Non-Patent Literatures 2 and 3 are ones reflecting fatigue resulting from a temporary load, and no diagnosis/evaluation of fatigue continuing for a long term in daily life has been established.

The present invention was accomplished in view of the above problems, and an object of the present invention is to provide a biomarker which enables objective and easy diagnosis and evaluation of fatigue.

### Solution to Problem

A first method of the present invention for evaluating fatigue includes at least two steps selected from the group consisting of: (1) obtaining a first ratio of a first measured value to a first standard value, the first measured value being a measured value of a glucose concentration in a biological sample obtained from a subject; (2) obtaining a second ratio of a second measured value to a second standard value, the second measured value being a measured value of a citrate concentration in the biological sample obtained from the subject; (3) obtaining a third ratio of a third measured value to a third standard value, the third measured value being a measured value of a cis-aconitate concentration in the biological sample obtained from the subject; (4) obtaining a fourth ratio of a fourth measured value to a fourth standard value, the fourth measured value being a measured value of an isocitrate concentration in the biological sample obtained from the subject; (5) obtaining a fifth ratio of a fifth measured value to a fifth standard value, the fifth measured value being a measured value of a succinate concentration in the biological sample obtained from the subject; (6) obtaining a sixth ratio of a sixth measured value to a sixth standard value, the sixth measured value being a measured value of a malate concentration in the biological sample obtained from the subject; and (7) obtaining a seventh ratio of a seventh measured value to a seventh standard value, the seventh measured value being a measured value of a lactate concentration in the biological sample obtained from the subject.

Diagnosis of chronic fatigue syndrome is confronted by problems such as many items to be measured until a diagnostic outcome is obtained, a long observation (measurement) time, an economic burden, and differences in judgment criterion among individual physicians. With the arrangement, the method of the present invention can provide data for making an objective diagnosis just by measuring specific metabolites in a biological sample. This allows for speedy, inexpensive, and objective diagnosis of fatigue. That is, the method of the present invention can be a method for providing a diagnostic criterion or an assessment criterion for fatigue. Moreover, since glucose, citrate, and cis-aconitate are deeply involved in energy production, the method of the present invention can be used for diagnosis of a wide variety of fatigue (including chronic fatigue, accumulated fatigue, and chronic fatigue syndrome).

The term “biological sample” used herein refers to any tissue (including a body fluid such as blood) or any cell taken from a subject, and encompasses also a tissue slice prepared from such a tissue and a cell lysate prepared from such a cell. Examples of a preferable biological sample for use in the present invention encompass, but are not limited to, blood, saliva, urine, interstitial fluid, sweat, and preparation (e.g., blood serum, blood prism) prepared from these. Note that it is a physician who carries out, as the first stage of obtaining a sample, a step of taking a tissue or a cell directly from a human subject, and therefore such a step is outside the scope of the present invention. Similarly, a step in which a physician assesses, on the basis of a result obtained by the method of the present invention, whether or not a subject is suffering from fatigue (including chronic fatigue, accumulated fatigue, and chronic fatigue syndrome) is outside the scope of the present invention.

The first through seventh standard values as used herein are preferably an average of glucose concentrations, an average of citrate concentrations, an average of cis-aconitate concentrations, an average of isocitrate concentrations, an average of succinate concentrations, an average of malate concentrations, and an average of lactate concentrations in respective biological samples obtained from a plurality of healthy subjects, respectively, but are not limited to such average values and can be modes obtained by binominal distribution or the like. The first through seventh standard values may be predefined values or may be values calculated on the basis of concentrations of metabolites in biological samples that are obtained from healthy subjects at the same time as sampling from a subject. The first through seventh standard values are preferably obtained by a same measuring method to that used to obtain the first through seventh measured values.

The first method of the present invention for evaluating fatigue may further include the step of comparing at least one pair of ratios out of at least two ratios obtained by the at least two steps. The first method of the present invention for evaluating fatigue preferably includes at least two steps selected from the group consisting of the steps (1) through (5). The comparison between the at least one pair of ratios may be one for obtaining a difference between the two values or may be one for obtaining a ratio of the two values.

In one aspect, the first method of the present invention for evaluating fatigue further includes at least one of (a) the step of comparing the first ratio and the second ratio, (b) the step of comparing the second ratio and the third ratio, and (c) the step of comparing the first ratio and the third ratio, and may further include the step of assessing whether or not at least one of the following conditions (I) through (III) is satisfied: (I) the first ratio is larger than the second ratio, (II) the second ratio is larger than the third ratio, and (III) the first ratio is larger than the third ratio.

In another aspect, the first method of the present invention for evaluating fatigue further includes at least one of (d) the step of comparing the first ratio and the fourth ratio, (e) the step of comparing the second ratio and the fourth ratio, and (f) the step of comparing the third ratio and the fourth ratio, and may further include the step of assessing whether or not at least one of the following conditions (IV) through (VI) is satisfied: (IV) the first ratio is larger than the fourth ratio, (V) the second ratio is larger than the fourth ratio, and (VI) the fourth ratio is larger than the third ratio. In a case where the first method of the present invention for evaluating fatigue includes the step (4), the first method of the present invention for evaluating fatigue preferably includes the step (5). In this case, the method of the present invention for evaluating fatigue further includes at least one of (g) the step of comparing the first ratio and the fifth ratio, (h) the step of comparing the second ratio and the fifth ratio, (i) the step of comparing the third ratio and the fifth ratio, and (j) the step of comparing the fourth ratio and the fifth ratio, and may further include the step of assessing whether or not at least one of the following conditions (VII) through (IX) is satisfied: (VII) the first ratio is larger than the second ratio, (VIII) the second ratio is larger than the fourth ratio, and (IX) the fifth ratio is larger than the fourth ratio.

Succinate is also deeply involved in energy production as with glucose, citrate, and cis-aconitate, and therefore can be used also for diagnosis of a wide variety of fatigue. Although cis-aconitate is unstable and its concentration in a sample is not easy to measure, use of a succinate concentration in a sample makes it possible to more easily carry out the present invention.

In a case where ratios are obtained in the steps (a) through (j), the first method of the present invention for evaluating fatigue may further include the step of carrying out an analysis such as a discriminant analysis, Partial Least Square, or Support Vector Machine with the use of at least one of the ratios thus obtained. The sixth ratio obtained in the step (6) and the seventh ratio obtained in the step (7) can be used just like the first through fifth ratios. Malate and lactate also are deeply involved in energy production as with glucose, citrate, cis-aconitate, and succinate, and therefore can be used also for diagnosis of a wide variety of fatigue.

With the arrangement, the method of the present invention can provide speedy, inexpensive, and objective diagnosis for chronic fatigue. Further, since a result of diagnosis made by use of the present invention reflects a clinical state itself, use of the present invention makes it possible to decide courses of treatment. That is, use of the present invention makes it possible to assess (or provide data for assessing) not only whether or not a subject is in a fatigue state, but also whether or not a subject is suffering from chronic fatigue and even whether or not a subject is suffering from chronic fatigue syndrome.

A second method of the present invention for evaluating fatigue includes at least one step selected from the group consisting of the steps (2) through (7). The second method of the present invention for evaluating fatigue may further include the steps of comparing the second through seventh ratios with corresponding reference values. Specifically, the second method of the present invention for evaluating fatigue may further include at least one of the following steps corresponding to the steps (2) through (7): (k) the step of comparing the second ratio with a reference value corresponding to the second ratio, (l) the step of comparing the third ratio with a reference value corresponding to the third ratio, (m) the step of comparing the fourth ratio with a reference value corresponding to the fourth ratio, (n) the step of comparing the fifth ratio with a reference value corresponding to the fifth ratio, (o) the step of comparing the sixth ratio with a reference value corresponding to the sixth ratio, and (p) the step of comparing the seventh ratio with a reference value corresponding to the seventh ratio. Note that each of the reference values is obtained as a ratio of a threshold value to a standard value, which threshold value is obtained through an analysis based on a decision tree mathematical model using a measured value. Specifically, each of the reference values is obtained as follows:

(threshold value/standard value)×100(%)

In this case, the second method of the present invention for evaluating fatigue preferably further includes the step of assessing whether or not at least one of the following conditions (X) through (XV) that correspond to the steps (k) through (p), respectively is satisfied: (X) the second ratio is smaller than the reference value corresponding to the second ratio, (XI) the third ratio is smaller than the reference value corresponding to the third ratio, (XII) the fourth ratio is smaller than the reference value corresponding to the fourth ratio, (XIII) the fifth ratio is smaller than the reference value corresponding to the fifth ratio, (XIV) the sixth ratio is smaller than the reference value corresponding to the sixth ratio, and (XV) the seventh ratio is smaller than the reference value corresponding to the seventh ratio.

In a case where at least one of the conditions (X) through (XV) is satisfied, a physician can assess that there is a possibility that the subject who provides the biological sample is suffering from chronic fatigue syndrome.

In a later-described system for evaluating fatigue, an assessing section can make a similar assessment. The second method of the present invention for evaluating fatigue is preferably used in a case where there is no substantial difference between the first measured value and the first standard value (i.e., the first ratio obtained in the step (1) is substantially equal to 1). In the present specification, objectively judging that there is no substantial difference between the first measured value and the first reference value without the use of the step (1) is also encompassed by judging that the first ratio is substantially equal to 1 with the use of the step (1).

In one aspect, the second method of the present invention for evaluating fatigue preferably includes at least one of the step of obtaining a third ratio, the step of obtaining a fourth ratio, and the step of obtaining a fifth ratio. As shown in FIGS. 3 and 5, there is a clear difference in terms of the third through fifth ratios between healthy subjects and patients with chronic fatigue syndrome. As described in Examples that will be described later, cis-aconitate, isocitrate, and succinate can be, by itself, a marker for assessing with 95% probability whether or not a subject is suffering from chronic fatigue syndrome. Succinate is most preferable as a marker, followed by isocitrate and cis-aconitate. That is, in the second method of the present invention for evaluating fatigue, the step of obtaining the fifth ratio is preferably carried out, the step of obtaining the fifth ratio and the step of obtaining the fourth ratio are more preferably carried out in this order, and the step of obtaining the fifth ratio, the step of obtaining the fourth ratio, and the step of obtaining the third ratio are most preferably carried out in this order.

A method of the present invention for evaluating fatigue includes at least one step selected from the group consisting of (1′) the step of comparing a measured value of a glucose concentration in a biological sample obtained from a subject with a first threshold value; (2′) the step of comparing a measured value of a citrate concentration in the biological sample obtained from the subject with a second threshold value; (3′) the step of comparing a measured value of a cis-aconitate concentration in the biological sample obtained from the subject with a third threshold value; (4′) the step of comparing a measured value of an isocitrate concentration in the biological sample obtained from the subject with a fourth threshold value; (5′) the step of comparing a measured value of a succinate concentration in the biological sample obtained from the subject with a fifth threshold value; (6′) the step of comparing a measured value of a malate concentration in the biological sample obtained from the subject with a sixth threshold value; and (7′) the step of comparing a measured value of a lactate concentration in the biological sample obtained from the subject with a seventh threshold value. The method of the present invention for evaluating fatigue preferably includes at least one of the steps (3′) through (5′), more preferably includes at least the step (5′), still more preferably includes the step (5′) and the step (4′) that are carried out in this order, and further more preferably includes the step (5′), the step (4′), and the step (3′) that are carried out in this order. The method of the present embodiment for evaluating fatigue may further include the step of assessing whether or not the following condition is satisfied in the steps (1′) through (7′): a measured value is smaller than a corresponding threshold value. From the viewpoint of assessment with 95% or higher probability as to whether or not a subject is suffering from chronic fatigue syndrome, the method preferably includes the step of assessing whether or not the following condition is satisfied in the steps (3′) through (5′): a measured value is smaller than a corresponding threshold value. In this case, the method of the present embodiment for evaluating fatigue may further include the step of assessing whether or not at least one measured value is smaller than a corresponding threshold value. In a case where at least one measured value is smaller than a corresponding threshold value, a physician can assess that there is a possibility that a subject who provides the biological sample is suffering from chronic fatigue syndrome. In a later-described system for evaluating fatigue, an assessing section can make a similar assessment.

A third method of the present invention for evaluating fatigue does not use the standard values, and includes the step of obtaining a ratio of at least one pair of measured values out of at least two measured values selected from the group consisting of first through fifth measured values in a biological sample obtained from a subject. The third method of the present invention for evaluating fatigue preferably further includes the step of subjecting the ratio of the at least one pair of measured values to a discriminant analysis, Partial Least Square, or Support Vector Machine. For example, in the method of the present invention for evaluating fatigue, it is preferable that the analysis is carried out with the use of at least one of (A) a ratio of the second measured value to the first measured value in the biological sample obtained from the subject, (B) a ratio of the third measured value to the first measured value in the biological sample obtained from the subject, (C) a ratio of the third measured value to the second measured value in the biological sample obtained from the subject, (D) a ratio of the fourth measured value to the first measured value, (E) a ratio of the fourth measured value to the second measured value, and (F) a ratio of the fourth measured value to the third measured value. With the arrangement, the method of the present invention makes it possible to identify a patient with chronic fatigue syndrome with 90% or higher accuracy. For example, in the method of the present invention for evaluating fatigue, it is more preferable that the analysis is carried out with the use of at least one of (i) a ratio of the second measured value to the first measured value in the biological sample obtained from the subject, (ii) a ratio of the fourth measured value to the first measured value in the biological sample obtained from the subject, (iii) a ratio of the fourth measured value to the second measured value in the biological sample obtained from the subject, (iv) a ratio of the fifth measured value to the first measured value, (v) a ratio of the fifth measured value to the second measured value, and (vi) a ratio of the fifth measured value to the fourth measured value. With the arrangement, the method of the present invention makes it possible to identify a patient with chronic fatigue syndrome with 90% or higher accuracy and 95% or higher sensitivity/specificity.

In the present invention, the steps of measuring a glucose concentration, a citrate concentration, a cis-aconitate concentration, an isocitrate concentration, a succinate concentration, a malate concentration, and a lactate concentration in a biological sample (the steps of obtaining first through seventh measured values) are not essential. The “measured values” to be obtained may be obtained and/or calculated by a person himself or herself who executes the present invention or may be obtained and/or calculated by a third party and then provided to a person who executes the present invention.

In order to evaluate fatigue, a first kit of the present invention includes a fifth reagent for measuring a succinate concentration. The first kit of the present invention may further include a fourth reagent for measuring an isocitrate concentration. The first kit of the present invention may further include at least one reagent selected from the group consisting of a first reagent for measuring a glucose concentration, a second reagent for measuring a citrate concentration, a third reagent for measuring a cis-aconitate concentration, a sixth reagent for measuring a malate concentration, and a seventh reagent for measuring a lactate concentration. The kit of the present invention preferably further includes a written direction describing standard values for a glucose concentration, a citrate concentration, a cis-aconitate concentration, an isocitrate concentration, a succinate concentration, a malate concentration, and a lactate concentration.

In order to evaluate fatigue, a second kit of the present invention includes a fifth presentation section which presents a fifth ratio. The second kit of the present invention may further include a fourth presentation section which presents a fourth ratio. The second kit of the present invention may further include at least one presentation section selected from the group consisting of a first presentation section which presents a first ratio, a second presentation section which presents a second ratio, a third presentation section which presents a third ratio, a sixth presentation section which presents a sixth ratio, and a seventh presentation section which presents a seventh ratio.

With the arrangement, the kit of the present invention can be easily used in actual medical practice It is therefore possible to provide a more speedy, less expensive, and objective method for evaluating fatigue and a more speedy, less expensive, and objective method for diagnosing chronic fatigue syndrome.

A first system of the present invention for evaluating fatigue includes a measured value receiving section which receives at least two measured values selected from the group consisting of first through seventh measured values; a standard value storage section in which at least two standard values selected from the group consisting of first through seventh standard values are stored; a computing section which receives (A) a measured value from the measured value receiving section and (B) a standard value from the standard value storage section and generates information for evaluating fatigue; and an evaluating section which receives the information from the computing section and evaluates fatigue, the computing section calculating at least two ratios selected from the group consisting of first through seventh ratios.

The first system of the present invention for evaluating fatigue is arranged such that the computing section executes a comparison between at least one pair of ratios out of the at least two ratios. The first system of the present invention for evaluating fatigue is preferably arranged such that the measured value receiving section receives at least two measured values selected from the group consisting of the first through fifth measured values, the standard value storage section stores therein at least two standard values selected from the group consisting of the first through fifth standard values, and the computing section further calculates at least two ratios selected from the group consisting of the first through fifth ratios and executes a comparison between at least one pair of ratios out of the at least two ratios. The comparison between the at least one pair of ratios may be one for obtaining a difference between the two values or may be one for obtaining a ratio of the two values. For example, the first system of the present invention for evaluating fatigue is preferably arranged such that the evaluating section assesses whether or not at least one of the following conditions (I) through (IX) is satisfied: (I) the first ratio is larger than the second ratio, (II) the second ratio is larger than the third ratio, (III) the first ratio is larger than the third ratio, (IV) the first ratio is larger than the fourth ratio, (V) the second ratio is larger than the fourth ratio, (VI) the fourth ratio is larger than the third ratio, (VII) the first ratio is larger than the second ratio, (VIII) the second ratio is larger than the fourth ratio, and (IX) the fifth ratio is larger than the fourth ratio. In a case where the comparison between the at least one pair of ratios is one for obtaining a ratio of the two values, the computing section may execute a discriminant analysis, Partial Least Square, or Support Vector Machine on the basis of the ratio thus obtained. The sixth ratio and the seventh ratio can be processed just like the first through fifth ratios in the first system of the present invention for evaluating fatigue.

A second system of the present invention for evaluating fatigue is arranged such that the computing section calculates a first ratio and at least one ratio selected from the group consisting of second through seventh ratios. The second system of the present invention for evaluating fatigue may be arranged such that the standard value storage section stores at least one reference value selected from the group consisting of reference values respectively corresponding to the second through seventh ratios. In this case, the computing section preferably receives the at least one reference value from the standard value storage section and executes a comparison between the calculated ratio and a reference value corresponding to the calculated ratio. Note that each of the reference values is obtained as a ratio of a threshold value to a standard value, which threshold value is obtained through an analysis based on a decision tree mathematical model using a measured value. Specifically, each of the reference values is obtained as follows:

(threshold value/standard value)×100(%)

In this case, the evaluating section assesses that the subject who provides the biological sample is suffering from chronic fatigue syndrome, in a case where at least one of the following conditions (A) through (F) is satisfied: (A) the second ratio is smaller than a reference value corresponding to the second ratio, (B) the third ratio is smaller than a reference value corresponding to the third ratio, (C) the fourth ratio is smaller than a reference value corresponding to the fourth ratio, (D) the fifth ratio is smaller than a reference value corresponding to the fifth ratio, (E) the sixth ratio is smaller than a reference value corresponding to the sixth ratio, and (F) the seventh ratio is smaller than a reference value corresponding to the seventh ratio. The second system of the present invention for evaluating fatigue is preferably arranged such that the computing section calculates at least one of the third through fifth ratios. In this case, the computing section preferably calculates the fifth ratio first, and then calculates the fourth ratio, and the computing section more preferably calculates the third ratio subsequently to the fourth ratio. The second system of the present invention for evaluating fatigue is preferably arranged such that the assessing section assesses whether or not the first ratio is substantially 1. With the arrangement, the system of the present invention makes it possible to identify a patient with chronic fatigue syndrome with 90% or higher accuracy and 95% or higher sensitivity/specificity.

The system of the present invention for evaluating fatigue is arranged such that the computing section compares the first through seventh measured values with threshold values respectively corresponding to the first through seventh measured values. The system of the present invention for evaluating fatigue may be arranged such that at least one threshold value selected from the group consisting of the threshold values respectively corresponding to the first through seventh measured values is stored in the standard value storage section. In the case, the computing section preferably receives the at least one threshold value from the standard value storage section and executes a comparison between a measured value and a threshold value corresponding to the measured value. The computing section preferably executes comparisons concerning the third through fifth measured values, more preferably executes a comparison concerning the fifth measured value first, further more preferably executes a comparison concerning the fifth measured value, a comparison concerning the fourth measured value, and a comparison concerning the third measured value in this order. From the viewpoint of assessment with 95% or higher probability as to whether or not a subject is suffering from chronic fatigue syndrome, the assessing section preferably assesses whether or not the following condition is satisfied in the steps (3′) through (5′): a measured value is smaller than a corresponding threshold value. In a case where at least one measured value is smaller than a corresponding threshold value, it is determined that a subject who provides the biological sample is suffering from chronic fatigue syndrome.

A third system of the present invention for evaluating fatigue includes a measured value receiving section which receives at least two measured values selected from the group consisting of first through fifth measured values; a computing section which receives a measured value from the measured value receiving section and generates information for evaluating fatigue; and an evaluating section which receives the information from the computing section and evaluates fatigue, the computing section executing a comparison between at least one pair of measured values out of the at least two measured values so as to obtain a ratio of the at least one pair of measured values. The third system of the present invention for evaluating fatigue is preferably arranged such that the computing section executes a discriminant analysis, Partial Least Square, or Support Vector Machine on the basis of the ratio of the at least one pair of measured values. With the arrangement, the system of the present invention can make an assessment with 90% or higher accuracy.

In a preferred aspect, the third system of the present invention for evaluating fatigue evaluates fatigue with the use of at least one of third, fourth, and fifth measured values in a biological sample obtained from a subject. The system of the present invention for evaluating fatigue includes a measured value receiving section which receives at least one measured value selected from the group consisting of the third, fourth and fifth measured values; a standard value storage section in which at least one standard value selected from the group consisting of third, fourth, and fifth standard values that respectively correspond to a cis-aconitate concentration, an isocitrate concentration, and a succinate concentration; a computing section which receives a measured value from the measured value receiving section and a standard value from the standard value storage section and generates information for evaluating fatigue; and an evaluating section which receives the information from the computing section and evaluates fatigue, the computing section calculating at least one of a third ratio, fourth ratio, and a fifth ratio. In this case, the computing section preferably calculates the fifth ratio, more preferably calculates the fifth ratio and the fourth ratio in this order, most preferably calculates the fifth ratio, the fourth ratio, and the third ratio in this order. The third system of the present invention for evaluating fatigue may be arranged such that the standard value storage section stores at least one reference value selected from the group consisting of reference values respectively corresponding to a cis-aconitate concentration, an isocitrate concentration, and a succinate concentration. In this case, the computing section preferably receives the at least one reference value from the standard value storage section and executes a comparison between a calculated ratio and a reference value corresponding to the calculated ratio. The computing section may, for example, carry out an analysis using at least one of the third ratio, the fourth ratio, and the fifth ratio.

The system of the present invention for evaluating fatigue may further include first through seventh measuring sections which measure the glucose concentration, the citrate concentration, the cis-aconitate concentration, the isocitrate concentration, the succinate concentration, the malate concentration, and the lactate concentration, respectively. In this case, the measured value receiving section receives values obtained by the first through seventh measuring sections.

With the arrangement, the system of the present invention uses a unique procedure originally found by the inventors to process measured values that can be obtained by a conventionally known easy technique. It is therefore possible to speedily present, at a low cost, an objective and highly reliable assessment result that cannot be obtained by a conventional art.

A suitable compound for use in the present invention is glucose, and it is preferable that at least one compound selected from the group consisting of citrate, cis-aconitate, isocitrate, succinate, malate, and lactate is used in combination with glucose. Since a citrate concentration, a succinate concentration, a malate concentration, and a lactate concentration are relatively easy to measure as compared with a cis-aconitate concentration and an isocitrate concentration, it is more preferable that at least one compound selected from the group consisting of citrate, succinate, malate, and lactate is used in combination of glucose, and it is also preferable that at least two compounds selected from the group consisting of citrate, succinate, malate, and lactate are used in combination of glucose.

As described above, in the present invention, in a case where two biomarkers are used, it is preferable that glucose is combined with one compound selected from the group consisting of citrate, cis-aconitate, isocitrate, succinate, malate, and lactate. In a case where three biomarkers are used, it is preferable that a combination of glucose, citrate, and succinate, a combination of glucose, citrate, and malate, a combination of glucose, citrate, and lactate, a combination of glucose, succinate, and malate, a combination of glucose, succinate, and a lactate, or a combination of glucose, malate, and lactate is used. In a case where four biomarkers are used, it is preferable that a combination of glucose, citrate, succinate, and malate, a combination of glucose, citrate, succinate, and lactate, a combination of glucose, citrate, malate, and lactate, a combination of glucose, succinate, malate, and lactate, or a combination of glucose, citrate, succinate, malate, and lactate is used.

### Advantageous Effects of Invention

The present invention makes it possible to objectively and easily evaluate fatigue and diagnose chronic fatigue syndrome. Further, the present invention can provide a technique useful for treatment of fatigue.

### BRIEF DESCRIPTION OF DRAWINGS

FIG. 1 is a diagram showing concentrations of various metabolites contained in blood plasma of a healthy subject and in blood plasma of a patient with chronic fatigue syndrome (CFS).

FIG. 2 is a diagram showing a result of a study on correlations between metabolites each forming a glycolytic system and a citric acid cycle and on correlations between the metabolites and PS (performance status).

FIG. 3 is a diagram showing a result of a study on amounts of candidate substances contained in blood plasma of healthy subjects and in blood plasma of patients with chronic fatigue syndrome (CFS). In FIG. 3, an average value of amounts of each candidate substance in the healthy subjects is set to 100, and an amount of each candidate substance is shown as a ratio to the average value 100.

FIG. 4 is a diagram showing, on three axes, a ratio of citrate to glucose, a ratio of cis-aconitate to citrate, and a ratio of isocitrate to cis-aconitate in blood plasma of the healthy subjects and in blood plasma of the patients with chronic fatigue syndrome (CFS).

FIG. 5 is a diagram showing a result of a study on amounts of candidate substances contained in blood plasma of healthy subjects and in blood plasma of patients with chronic fatigue syndrome (CFS). In FIG. 5, an average value of amounts of each candidate substance in the healthy subjects is set to 100, and an amount of each candidate substance is shown as a ratio to the average value 100.

FIG. 6 is a diagram showing, on three axes, a ratio of citrate to glucose, a ratio of isocitrate to citrate, and a ratio of succinate to isocitrate in blood plasma of the healthy subjects and in blood plasma of the patients with chronic fatigue syndrome (CFS).

FIG. 7 is a diagram showing a result of a Random Forest program using all parameters including measured values of metabolites in a blood plasma sample.

### DESCRIPTION OF EMBODIMENTS

[1] Fatigue

Currently, the number of patients who visit a medical institution with a main symptom of feeling of fatigue/weariness is second largest next to the number of patients whose main symptom is pain. However, no method for objectively evaluating fatigue has been developed to date. Feeling of fatigue and weariness is something which people experience in daily life, and is an important biological signal for warning disturbance of homeostasis in their bodies. However, feeling of fatigue is merely a subjective perception, and is not an objective indicator of a fatigue level. In the past, lactate was considered as a cause for fatigue, but it is becoming clear that a change of a lactate level cannot be an indicator of a fatigue status although it can be an indicator of exercise. Further, no effective treatment for fatigue has been found, and all known to date is that caffeine, taurine, ascorbic acid, etc. are anti-fatigue components.

Since the Centers for Disease Control and Prevention (CDC)'s report in 1988 of a disease, called “chronic fatigue syndrome”, which brings about fatigue of unknown cause, various researches centering on investigation into a mechanism of the disease, search for a biomarker, and development of a treatment/prevention method have been conducted. However, a mechanism of how chronic fatigue syndrome occurs has not been discovered yet, and no objective biomarker has been developed. Even now, a diagnosis of chronic fatigue syndrome is conducted by using a symptom and physical finding standard presented by CDC in 1994.

Meanwhile, fatigue biomarkers using virus activation or autonomic defect as an indicator have been proposed. However, these biomarkers are not based on a cause and a mechanism of human fatigue, and cannot be regarded as biomarkers specific to fatigue (especially chronic fatigue, chronic fatigue syndrome). Further, these biomarkers are indirect indicators of a mechanism of maintenance/recovery of homeostasis, but are not direct indicators of such a mechanism. This requires a process of trial and error before development of a specific treatment method.

A change of a blood amino acid amount (especially an increase in branched-chain amino acid amount) was found in a fatigue-load model animal. In view of this, the inventors of the present invention conducted a similar test by using blood of a fatigue patient, but could not obtain a similar result. To verify this result, the inventors of the present invention conducted a metabolomic analysis, which enables exhaustive analysis of various metabolites, by using blood plasma of a patient with chronic fatigue syndrome. However, no quantitative difference in branched-chain amino acid could be found between a healthy subject and the patient with chronic fatigue syndrome.

In researches using model animals, a decrease of ATP in the liver and muscle tissue, activation of brain cytokine, etc. have been reported, but application of such findings to diagnosis and/or treatment of a human is difficult from an ethical perspective. Since such findings obtained from model animals are far from application to fatigue patients, causes and a mechanism of fatigue have not been discovered yet.

[2] Biomarker of the Present Invention

The inventors of the present invention found a biomarker for fatigue by further analyzing changes of amounts of metabolites in blood plasma of healthy subjects and in blood plasma of patients with chronic fatigue syndrome and by combining a unique method originally found by the inventors with measured values thus obtained. Based on this finding, the inventors of the present invention accomplished the present invention.

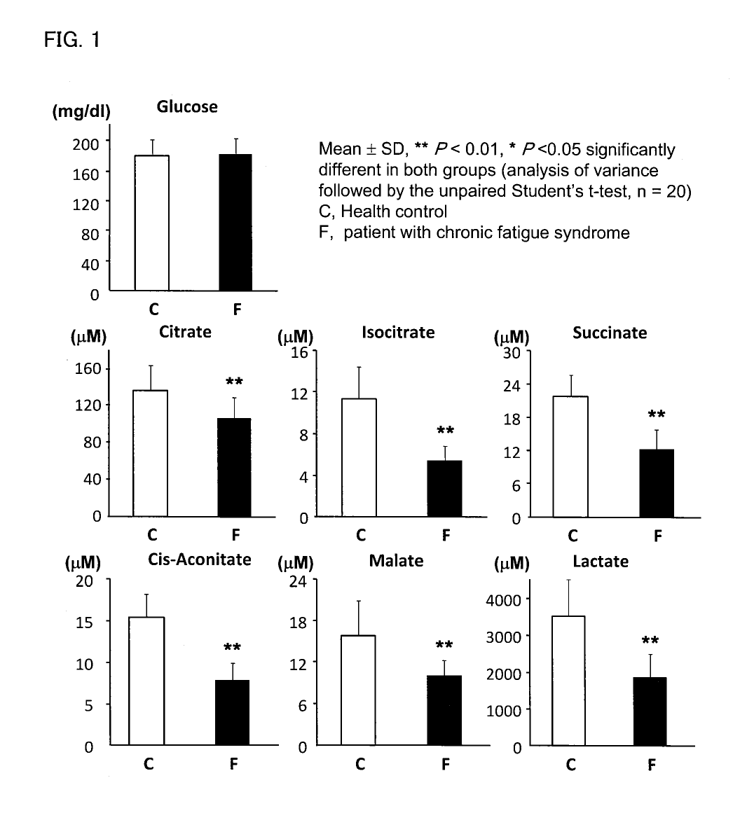

A biomarker of the present invention is based on a glucose concentration, a citrate concentration, a cis-aconitate concentration, an isocitrate concentration, a succinate concentration, a malate concentration, and a lactate concentration in a biological sample (see Examples that will be described later). In recent years, techniques for analyzing various items of various samples in an exhaustive manner have been developed. The metabolomic analysis adopted by the inventors of the present invention is one of them. However, there are a lot of compounds (metabolites) whose concentration in a biological sample significantly differs between a subject and a healthy subject (see FIG. 1), and no finding which clearly motivates selection of at least two (all, as needed) of the compounds (metabolites) exists in the art. The inventors of the present invention narrowed the metabolites whose concentration in a biological sample significantly differs between a subject and a healthy subject down to ones showing a significant correlation with PS, and subjected the metabolites showing a significant correlation with PS to various mathematical model analysis methods. This procedure is also a unique perspective of the inventors of the present invention. Both of (A) Selection of glucose, in addition to compounds obtained by the metabolomic analysis, as a compound to be subjected to a path analysis and (B) association of glucose with other metabolites are also unique perspectives of the inventors of the present invention. In a case of using the aforementioned metabolites (i.e., in a case of using at least two biomarkers), it is possible to produce a remarkably excellent effect of distinguishing a healthy subject and a patient with chronic fatigue syndrome with higher than 90% accuracy.

There have been known reports that associate citrate with fatigue (see, for example, Patent Literatures 2 and 3). However, a person skilled in the art cannot arrive at the present invention on the basis of any of these reports.

According to the present invention, ratios of measured values of metabolites (glucose, citrate, cis-aconitate, isocitrate, and succinate) in a biological sample of a subject to measured values of the metabolites in a biological sample of a healthy subject are calculated, and a difference between any two of the ratios thus calculated is used as a biomarker. For example, according to the present invention, a ratio (first ratio R₁) of a glucose concentration (measured value M₁) in a biological sample obtained from a subject and a standard value for glucose (first standard value B₁) is calculated, a ratio (second ratio R₂) of a citrate concentration (measured value M₂) in the biological sample obtained from the subject and a standard value for citrate (second standard value B₂) is calculated, and a ratio (third ratio R₃) of a cis-aconitate concentration (measured value M₃) in the biological sample obtained from the subject and a standard value for cis-aconitate (third standard value B₃) is calculated. By comparing these ratios R₁ through R₃, biomarkers of the present invention are obtained.

In First Embodiment, the biomarkers of the present invention can be a difference Q₁ between R₁ and R₂ and a difference Q₂ between R₁ and R₃.

In a case where at least one of the biomarkers Q₁ (biomarker Q₁=R₁−R₂) and Q₂ (biomarker Q₂=R₂−R₃) is positive (Q₁>0 or Q₂>0), it is determined that a subject is “in a fatigue state”. Meanwhile, in a case where both of the biomarkers Q₁ and Q₂ are negative (Q₁<0 and Q₂<0), it is determined that the subject is “not in a fatigue state”. Note that it is also possible that (A) a ratio of a measured value of isocitrate in a biological sample of a subject to a measured value of isocitrate in a biological sample of a healthy subject is calculated and (B) a difference between the isocitrate ratio and the cis-aconitate ratio is used as a biomarker. Specifically, it is also possible that (A) a ratio (fourth ratio R₄) of an isocitrate concentration (measured value M₄) in the biological sample obtained from the subject and a standard value for isocitrate (fourth standard value B₄) is calculated and (B) a biomarker Q₃ (biomarker Q₃=R₄−R₃) obtained based on the ratios R₃ and R₄ is used along with the biomarkers Q₁ and Q₂. In this case, in a case where at least one of the biomarkers Q₁ through Q₃ is positive (Q₁>0, Q₂>0 or Q₃>0), it is determined that the subject is “in a fatigue state”. Meanwhile, in a case where all of the biomarkers Q₁ through Q₃ are negative (Q₁<0, Q₂<0, and Q₃<0), it is determined that the subject is “not in a fatigue state”.

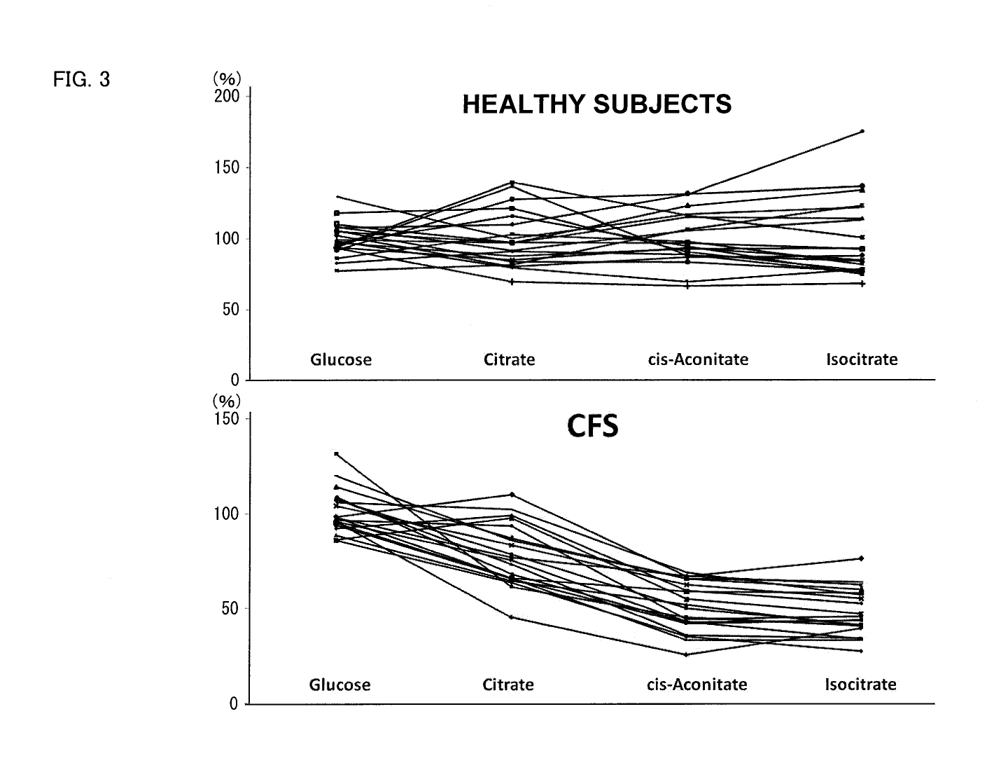

Such an embodiment is demonstrated in an Example (see, for example, FIG. 3) that will be described later. A person skilled in the art who read the present specification would easily understand that a biomarker Q₄ (biomarker Q₄=R₁−R₃), a biomarker Q₅ (biomarker Q₅=R₁−R₄), and a biomarker Q₆ (biomarker Q₆=R₂−R₄) are also biomarkers of the present invention as with the biomarkers Q₁ through Q₃ and can be used for evaluation of fatigue as with the biomarkers Q₁ through Q₃.

Since cis-aconitate is unstable, its concentration in a sample is not easy to measure. In such a case, succinate can be used along with glucose, citrate, and isocitrate. Succinate is also deeply involved in energy production as with glucose, citrate, and cis-aconitate. Use of a succinate concentration in a sample makes it possible to more easily carry out the present invention. Specifically, in First Embodiment, it is preferable that (A) a ratio (first ratio R₁) of a glucose concentration (measured value M₁) in a biological sample obtained from a subject and a standard value for glucose (first standard value B₁) is calculated, (B) a ratio (second ratio R₂) of a citrate concentration (measured value M₂) in the biological sample obtained from the subject and a standard value for citrate (second standard value B₂) is calculated, (C) a ratio (fourth ratio R₄) of an isocitrate concentration (measured value M₄) in the biological sample obtained from the subject and a standard value for isocitrate (fourth standard value B₄) is calculated, (D) a ratio (fifth ratio R₅) of a succinate concentration (measured value M₅) in the biological sample obtained from the subject and a standard value for succinate (fifth standard value B₅) is calculated, and (E) biomarkers of the present invention are obtained by comparing these ratios R₁, R₂, R₄, and R₅. In this case, the biomarkers of the present embodiment can be a difference Q₁ₐ between R₁ and R₂, a difference Q₂ₐ between R₂ and R₄, and a difference Q₃ₐ between R₄ and R₅.

In a case where at least one of the biomarkers Q₁ₐ (biomarker Q₁ₐ=R₁−R₂), Q₂ₐ (biomarker Q₂ₐ=R₂−R₄), and Q₃ₐ (biomarker Q₃ₐ=R₅−R₄) is positive (Q₁ₐ>0, Q₂ₐ>0 or Q₃ₐ>0), it is determined that the subject is “in a fatigue state”. Meanwhile, in a case where all of the biomarkers Q1a, Q₂ₐ, and Q₃ₐ is negative (Q₁ₐ<0 and Q₂ₐ<0 and Q₃ₐ<0), it is determined that the subject is “not in a fatigue state”.

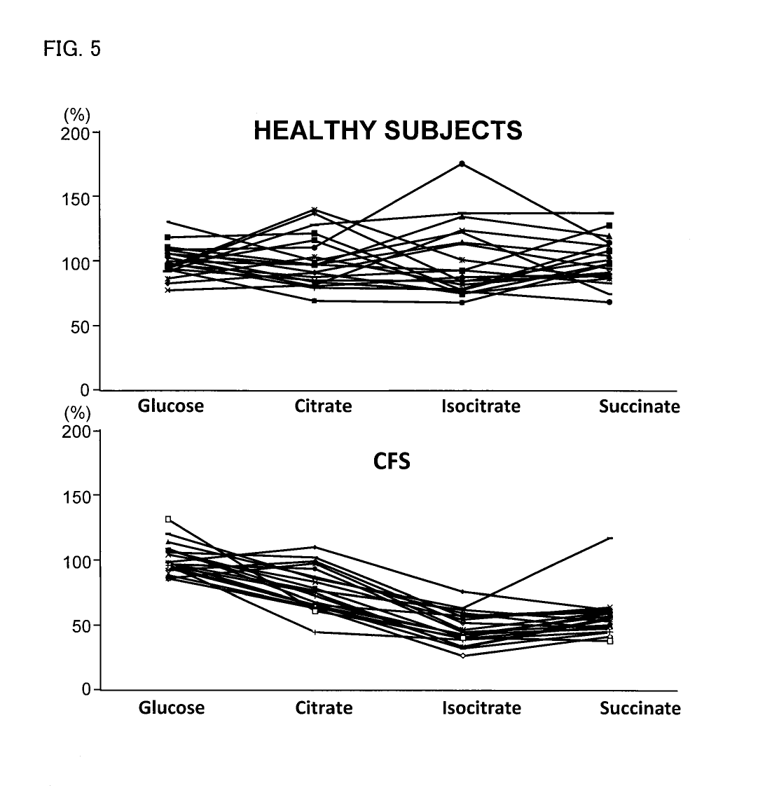

Such an embodiment is demonstrated in an Example (see, for example, FIG. 5) that will be described later. A person skilled in the art who read the present specification would easily understand that a biomarker Q₄ₐ (biomarker Q₄ₐ=R₁−R₄), a biomarker Q₅ₐ (biomarker Q₅ₐ=R₁−R₅), and a biomarker Q₆ₐ (biomarker Q₆ₐ=R₂−R₅) are also biomarkers of the present invention as with the biomarkers Q₁ₐ, Q₂ₐ, and Q₃ₐ, and can be used for evaluation of fatigue as with the biomarkers Q₁ₐ, Q₂ₐ, and Q₃ₐ.

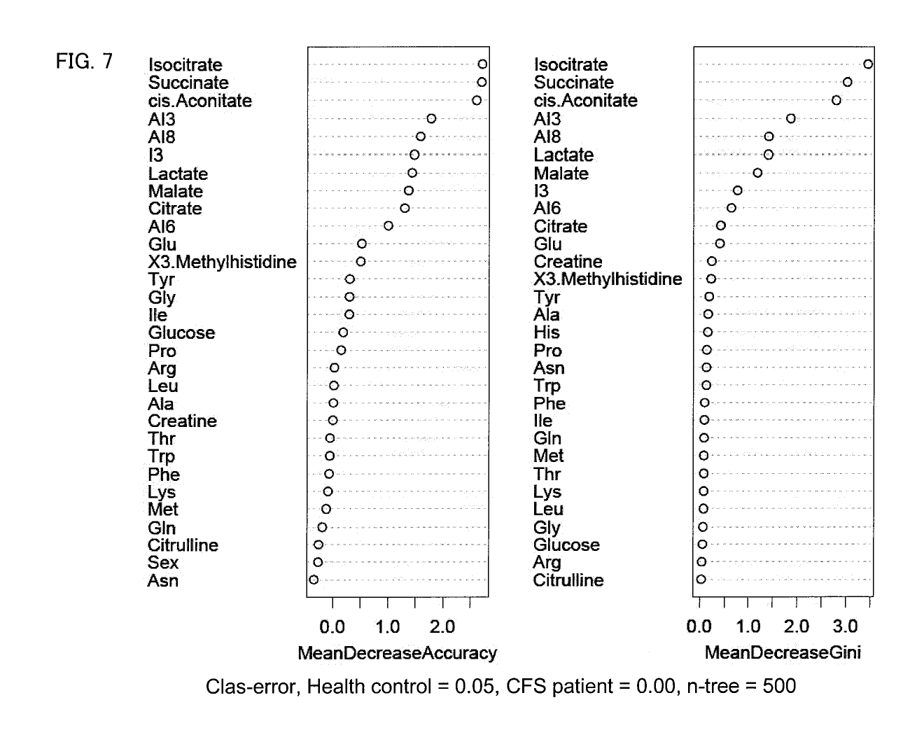

In a case where there is no substantial difference between the measured value of the glucose concentration in the biological sample obtained from the subject and the standard value (i.e., R₁≈1), R₂ through R₅ can be biomarkers. Further, it is possible that (A) a ratio (sixth ratio R₆) of a malate concentration (measured value M₆) in the biological sample obtained from the subject and a standard value for malate (sixth standard value B₆) is calculated, (B) a ratio (seventh ratio R₇) of a lactate concentration (measured value M₇) in the biological sample obtained from the subject and a standard value for lactate (seventh standard value B₇) is calculated, and (C) these ratios R₆ and R₇ are used along with R₁ through R₅. That is, in Second Embodiment, the biomarkers of the present invention can be R₂ through R₇. Further biomarkers of the present invention are provided as a biomarker Q₇ (biomarker Q₇=second ratio R₂), a biomarker Q₈ (biomarker Q₈=third ratio R₃), a biomarker Q₉ (biomarker Q₉=fourth ratio R₄), a biomarker Q₁₀ (biomarker Q₁₀=fifth ratio R₅), a biomarker Q₁₁ (biomarker Q₁₁=sixth ratio R₆), and a biomarker Q₁₂ (biomarker Q₁₂=seventh ratio R₇). As a result of a decision tree mathematical model analysis, each of the biomarkers (Q₇ through Q₁₂) shows 90% or higher sensitivity and specificity. According to an analysis using the items shown in FIG. 7, in a case where at least one of the biomarkers Q₇ through Q₁₂ satisfies a condition (R₂<79.1%, R₃<69.3%, R₄<66.0%, R₅<66.6%, R₆<83.7%, and R₇<74.3%) it is determined that the subject is “in a fatigue state”, whereas in a case where all of the biomarkers Q₇ through Q₁₂ do not satisfy the condition, it is determined that the subject is “not in a fatigue state” (see Examples that will be described later).

Cis-aconitate, isocitrate, and succinate each can be used alone, without being combined with another factor, to distinguish between a healthy subject and a patient (see Examples that will be described later). Such a very excellent function is a particularly remarkable effect that cannot be expected by a person skilled in the art.

Based on the findings obtained by the biomarkers, measured values themselves of a glucose concentration, a citrate concentration, a cis-aconitate concentration, an isocitrate concentration, and a succinate concentration in a biological sample can also provide a fatigue diagnosis/assessment standard (e.g., data for diagnosis or assessment of fatigue), and the measured values M₂ through M₇ of the concentrations of the metabolites in the biological sample obtained from the subject can also be biomarkers. That is, further biomarkers of the present invention are provided as a biomarker Q₇ₐ (biomarker Q₇ₐ=second measured value M₂), a biomarker Q₈ₐ (biomarker Q₈ₐ=third measured value M₃), a biomarker Q₉ₐ (biomarker Q₉ₐ=fourth measured value M₄), a biomarker Q₁₀ₐ (biomarker Q₁₀ₐ=fifth measured value M₅), a biomarker Q₁₁ₐ (biomarker Q₁₁ₐ=sixth measured value M₆), and a biomarker Q₁₂ₐ (biomarker Q₁₂ₐ=seventh measured value M₇). As a result of a decision tree mathematical model analysis, each of the biomarkers (Q₇ₐ through Q₁₂ₐ) shows 90% or higher sensitivity and specificity. Especially Q₈ₐ through Q₁₀ₐ are preferable, and it is possible that in a case where at least one of the biomarkers Q₈ₐ through Q₁₀ₐ satisfies a condition of being smaller than a corresponding threshold value, it is determined that the subject is “in a fatigue state”, whereas in a case where all of the biomarkers Q₈ₐ through Q₁₀ₐ do not satisfy the condition, it is determined that the subject is “not in a fatigue state”. It is preferable that (A) the biomarker Q₉ₐ be used in priority to the biomarkers Q₈ₐ and Q₁₀ₐ, (B) the biomarker Q₈ₐ be used next, and (C) then the biomarker Q₁₀ₐ be used. This makes it possible to assess, with 95% or higher probability, whether or not a subject is suffering from chronic fatigue syndrome.

As shown in Examples that will be described later, cis-aconitate, isocitrate, and succinate each can be used alone, without being combined with another factor, to distinguish between a healthy subject and a patient. Such a very excellent function is a particularly remarkable effect that cannot be expected by a person skilled in the art.

Further, biomarkers of the present invention can be obtained by comparing a glucose concentration (measured value M₁), a citrate concentration (measured value M₂), a cis-aconitate concentration (measured value M₃), an isocitrate concentration (measured value M₄), and a succinate concentration (measured value M₅) in a biological sample obtained from a subject. In Third Embodiment, biomarkers of the present invention are provided as a biomarker Q₁₃ (biomarker Q₁₃=M₂/M₁), a biomarker Q₁₄ (biomarker Q₁₄=M₃/M₂), a biomarker Q₁₅ (biomarker Q₁₅=M₄/M₃), a biomarker Q₁₆ (biomarker Q₁₆=M₃/M₁), a biomarker Q₁₇ (biomarker Q₁₇=M₄/M₁), and a biomarker Q₁₈ (biomarker Q₁₈=M₄/M₂), and these biomarkers Q₁₃ through Q₁₈ can be used similarly.

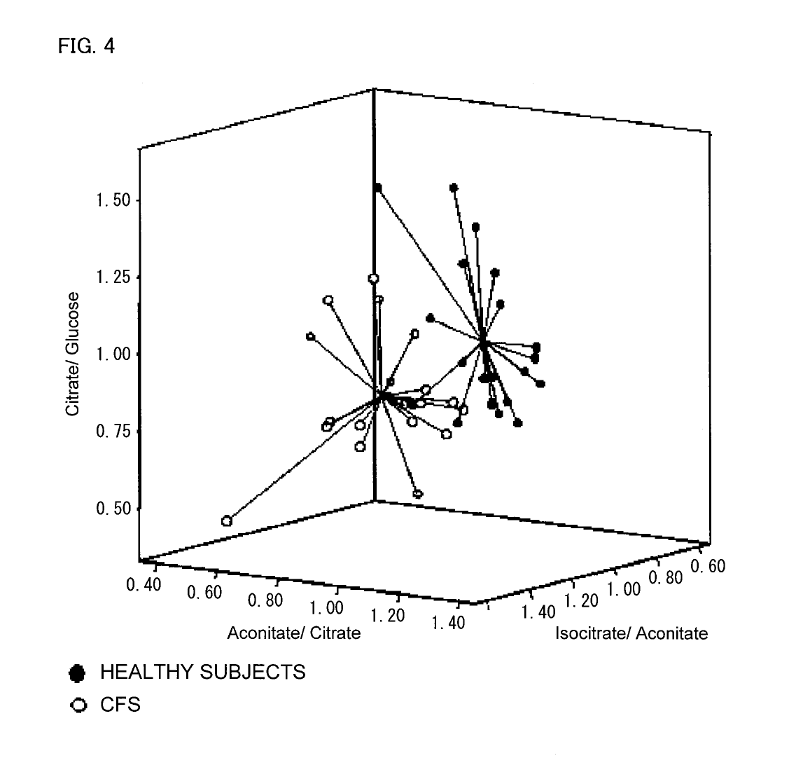

Such an embodiment is demonstrated in an Example (see, for example, FIG. 4) that will be described later. In a case where three of these biomarkers Q₁₃ through Q₁₈ are plotted triaxially as shown in FIG. 4, it is possible to distinguish a group of normal subjects and a group of patients with chronic fatigue syndrome. For example, as a result of calculation of values a, b, c, and d in the following formula (A) by a discriminant analysis with the use of Q₁₃ through Q₁₅ (see Examples that will be described later), patients with chronic fatigue syndrome could be identified with 90% or higher accuracy:

(a*Q₁₃+b*Q₁₄+c*Q₁₅)+d (A)

Here, a case where d>0 represents the normal group and a case where d<0 represents the chronic fatigue syndrome group. A similar result could be obtained even in a case where another analysis such as Partial Least Square or Support Vector Machine is carried out instead of the discriminant analysis.

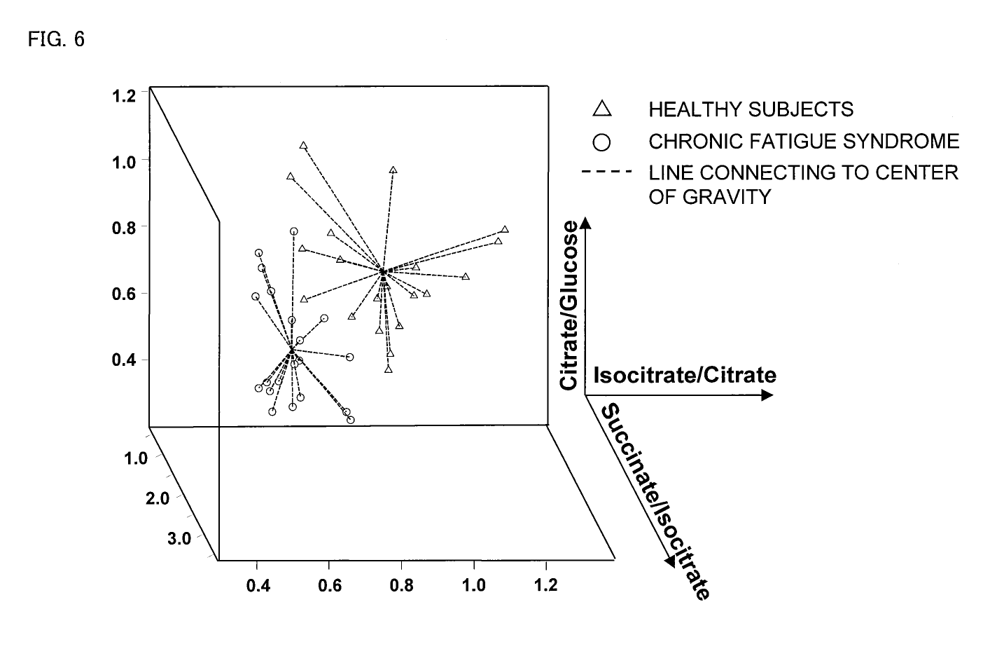

In the present embodiment, in a case where isocitrate is used along with glucose, citrate, and succinate, biomarkers of the present invention are similarly provided as a biomarker Q₁₃ₐ (biomarker Q₁₃ₐ=M₂/M₁), a biomarker Q₁₄ₐ (biomarker Q₁₄ₐ=M₄/M₂), a biomarker Q₁₅ₐ (biomarker Q₁₅ₐ=M₅/M₄), a biomarker Q₁₆ₐ (biomarker Q₁₆ₐ=M₄/M₁), a biomarker Q₁₇ₐ (biomarker Q₁₇ₐ=M₅/M₁), and a biomarker Q₁₈ₐ (biomarker Q₁₈ₐ=M₅/M₂), and these biomarkers Q₁₃ₐ through Q₁₈ₐ can be used similarly. In a case where three of these biomarkers Q₁₃ₐ through Q₁₈ₐ are plotted triaxially as shown in FIG. 6, it is possible to distinguish a normal group and a chronic fatigue syndrome group. For example, as a result of calculation of values a, b, c, and d in the following formula (A′) by a discriminant analysis with the use of Q₁₃ₐ through Q₁₅ₐ (see Examples that will be described later), patients with chronic fatigue syndrome could be identified with 90% or higher accuracy and 95% or higher sensitivity/specificity:

(a*Q₁₃ₐ+b*Q₁₄ₐ+c*Q₁₅ₐ)+d (A′)

Here, a case where d>0 represents the normal group and a case where d<0 represents the chronic fatigue syndrome group. A similar result could be obtained even in a case where another analysis such as Partial Least Square or Support Vector Machine is carried out instead of the discriminant analysis.

It is through accomplishment of the present invention that it has been sufficiently confirmed that glucose, citrate, cis-aconitate, isocitrate, and succinate are very useful factors for distinguishing between a healthy subject and a patient. That is, a person skilled in the art could not have easily found, based on conventional findings, that these factors are available for distinguishing between a healthy subject and a patient.

As described above, the biomarkers of the present invention allows for objective and easy evaluation and diagnosis of fatigue. Further, use of the biomarkers of the present invention makes it possible to develop diagnosis and treatment methods for chronic fatigue syndrome.

[3] Use of Biomarker of the Present Invention

The present invention provides a method, a kit, and a system for evaluating fatigue on the basis of the biomarkers.

[3-1] Method for Evaluating Fatigue

A fatigue status of a subject can be evaluated with the use of at least two of the biomarkers Q₁ through Q₆ of First Embodiment (or at least two of the biomarkers Q₁ₐ through Q₆ₐ). For example, the biomarkers Q₁ (Q₁=first ratio R₁−second ratio R₂) and Q₂ (Q₂=second ratio R₂−third ratio R₃) can be used. In a case where Q₁>0 or Q₂>0, it is determined that a subject is “in a fatigue state”, whereas in a case where Q₁<0 and Q₂<0, it is determined that the subject is “not in a fatigue state”. That is, a fatigue status of a subject can be evaluated by calculating Q₁ and Q₂ on the basis of the first through third ratios R₁ through R₃ (R₁=M₁/B₁, R₂=M₂/B₂, R₃=M₃/B₃). Note that there are cases where a fatigue status of a subject can be evaluated only by calculating one of Q₁ and Q₂ since it is only necessary that at least one of Q₁ and Q₂ be positive. That is, there are cases where a fatigue status of a subject can be evaluated only by comparing R₁ and R₂ or only by comparing R₂ and R₃.

From this perspective, in an embodiment, a method of the present invention for evaluating fatigue includes the steps of: (1) obtaining a first ratio of a measured value of a glucose concentration in a biological sample obtained from a subject to a first standard value; (2) obtaining a second ratio of a measured value of a citrate concentration in the biological sample obtained from the subject to a second standard value; (3) obtaining a third ratio of a measured value of a cis-aconitate concentration in the biological sample obtained from the subject to a third standard value, and further includes at least one of the steps of comparing the first ratio and the second ratio and comparing the second ratio and the third ratio. The method for evaluating fatigue in accordance with the present embodiment may further include the step of assessing whether or not at least one of the following conditions is satisfied: (I) the first ratio is larger than the second ratio, (II) the second ratio is larger than the third ratio, and (III) the first ratio is larger than the third ratio.

Use of the method of the present invention for evaluating fatigue makes it possible to not only evaluate fatigue and propose a treatment method, but also provide a judgment criterion for fatigue (including chronic fatigue and chronic fatigue syndrome). This makes it easier to make a diagnosis as to whether a subject is in a fatigue state or not. That is, the method of the present invention for evaluating fatigue can be a method for providing a diagnostic criterion or assessment criterion for fatigue (including chronic fatigue and chronic fatigue syndrome) (e.g., a method for acquiring data for diagnosing or assessing fatigue (including chronic fatigue and chronic fatigue syndrome)).

As described above, the biomarker Q₃ is also useful for evaluation of a fatigue status of a subject. In a case where the biomarker Q₃ (biomarker Q₃=fourth ratio R₄−third ratio R₃) is used in combination with the biomarkers Q₁ and Q₂, a fatigue status of a subject can be evaluated as follows. Specifically, in a case where Q₁>0, Q₂>0, or Q₃>0, it is determined that the subject is “in a fatigue state”, whereas in a case where Q₁<0 and Q₂<0 and Q₃<0, it is determined that the subject is “not in a fatigue state”. That is, a fatigue status of a subject can be evaluated by using Q₃ calculated on the basis of the fourth ratio R₄ (R₄=M₄/B₄) and the third ratio R₃, as in the case of using Q₁ and Q₂. That is, the method of the present invention for evaluating fatigue may further include the step of (4) obtaining a fourth ratio of a measured value of an isocitrate concentration in the biological sample obtained from the subject to a fourth standard value. In this case, the method of the present invention for evaluating fatigue preferably further includes at least one of the steps of comparing the first ratio and the fourth ratio, comparing the second ratio and the fourth ratio, and comparing the third ratio and the fourth ratio. For example, the method of the present invention for evaluating fatigue more preferably further includes the step of carrying out an analysis such as a discriminant analysis, Partial Least Square, or Support Vector Machine with the use of at least one of (a) a ratio of the second ratio to the first ratio, (b) a ratio of the third ratio to the second ratio, and (c) a ratio of the fourth ratio to the third ratio.

In the present embodiment, in a case where the fourth ratio R₄ concerning isocitrate is used, the fourth ratio R₄ is preferably used along with the first ratio R₁ concerning glucose, the second ratio R₂ concerning citrate, and the fifth ratio R₅ concerning succinate as described above. That is, biomarkers of the present invention are provided as the biomarker Q₁ₐ (biomarker Q₁ₐ=first ratio R₁−second ratio R₂), the biomarker Q₂ₐ (biomarker Q₂ₐ=second ratio R₂−fourth ratio R₄), and the biomarker Q₃ₐ (biomarker Q₃ₐ=fifth ratio R₅−fourth ratio R₄). In a case where at least one of Q₁ₐ>0, Q₂ₐ>0, and Q₃ₐ>0 is satisfied, it is determined that the subject is “in a fatigue state”, whereas in a case where Q₁ₐ<0 and Q₂ₐ<0 and Q₃ₐ<0 are satisfied, it is determined that the subject is “not in a fatigue state”. That is, a fatigue status of a subject can be evaluated by calculating Q₁ₐ through Q₃ₐ on the basis of the first ratio (R₁=M₁/B₁), the second (R₂=M₂/B₂), the fourth ratio (R₄=M₄/B₄), and the fifth ratio (R₅=M₅/B₅). Note that there are cases where a fatigue status of a subject can be evaluated only by calculating one of Q₁ₐ through Q₃ₐ since it is only necessary that at least one of Q₁ₐ through Q₃ₐ be positive.

From this perspective, in a preferred embodiment, a method of the present invention for evaluating fatigue includes at least two of the steps of: (1) obtaining a first ratio of a measured value of a glucose concentration in a biological sample obtained from the subject to a first standard value; (2) obtaining a second ratio of a measured value of a citrate concentration in the biological sample obtained from the subject to a second standard value; (3) obtaining a fourth ratio of a measured value of an isocitrate concentration in the biological sample obtained from the subject to a fourth standard value; and (4) obtaining a fifth ratio of a measured value of a succinate concentration in the biological sample obtained from the subject to a fifth standard value, and further includes at least one of the steps of comparing the first ratio and the second ratio, comparing the second ratio and the fourth ratio, and comparing the fourth ratio and the fifth ratio. The method for evaluating fatigue in accordance with the present embodiment may further include the step of assessing whether or not at least one of the following conditions is satisfied: (I) the first ratio is larger than the second ratio, (II) the second ratio is larger than the third ratio, and (III) the fourth ratio is larger than the third ratio. The method for evaluating fatigue in accordance with the present embodiment may further include the step of assessing whether or not at least one of the following conditions is satisfied: (IV) the first ratio is larger than the second ratio, (V) the second ratio is larger than the fourth ratio, and (VI) the fourth ratio is smaller than the fifth ratio.

Further, use of the biomarkers Q₇ through Q₁₂ of Second Embodiment makes it possible to evaluate (diagnose or assess) whether or not a subject is suffering from chronic fatigue syndrome. As described above, as a result of a decision tree mathematical model analysis, each of the biomarkers Q₇ through Q₁₂ shows 90% or higher sensitivity and specificity. Assume that the measured value of the glucose concentration in the biological sample obtained from the subject is substantially equal to the standard value. In this case, according to analysis using the items shown in FIG. 7, in a case where at least one of the biomarkers Q₇ through Q₁₂ satisfies a condition (R₂<79.1%, R₃<69.3%, R₄<66.0%, R₅<66.6%, R₆<83.7%, and R₇<74.3%), it is determined that the subject is “in a fatigue state”, whereas in a case where all of the biomarkers Q₇ through Q₁₂ do not satisfy the condition, it is determined that the subject is “not in a fatigue state” (see Examples that will be described later).

From this perspective, in a preferred embodiment, a method of the present invention for evaluating fatigue includes the step of (1) comparing a measured value of a glucose concentration in a biological sample obtained from a subject with a first standard value, and further includes at least one of the steps of: (2) obtaining a second ratio of a measured value of a citrate concentration in the biological sample obtained from the subject to a second standard value; (3) obtaining a third ratio of a measured value of a cis-aconitate concentration in the biological sample obtained from the subject to a third standard value; (4) obtaining a fourth ratio of a measured value of an isocitrate concentration in the biological sample obtained from the subject to a fourth standard value; (5) obtaining a fifth ratio of a measured value of a succinate concentration in the biological sample obtained from the subject to a fifth standard value; (6) obtaining a sixth ratio of a measured value of a malate concentration in the biological sample obtained from the subject to a sixth standard value; and (7) obtaining a seventh ratio of a measured value of a lactate concentration in the biological sample obtained from the subject to a seventh standard value. The method of the present invention for evaluating fatigue may further include the step of, in a case where it is determined in the step (1) that there is no substantial difference between the measured value of the glucose concentration and the first standard value, assessing whether or not the ratios obtained in the steps (2) through (7) are smaller than respective predetermined percentages. According to the analysis using the items shown in FIG. 7, the predetermined percentages for the ratios obtained in the steps (2) through (7) are 79.1%, 69.3%, 66.0%, 66.6%, 83.7%, and 74.3%, respectively (see Examples that will be described later). This makes it possible to assess, with 95% or higher probability, whether or not a subject is suffering from chronic fatigue syndrome.

Further, use of the biomarkers Q₇ₐ through Q₁₂ₐ makes it possible to evaluate (diagnose or assess) whether or not a subject is suffering from chronic fatigue syndrome. As described above, as a result of a decision tree mathematical model analysis, each of the biomarkers Q₇ₐ through Q₁₂ₐ shows 90% or higher sensitivity and specificity. Especially Q₈ₐ through Q₁₀ₐ are preferably used, and it is possible that in a case where at least one of the biomarkers Q₈ₐ through Q₁₀ₐ satisfies a condition of being smaller than a corresponding threshold value, it is determined that the subject is “in a fatigue state”, whereas in a case where all of the biomarkers Q₈ₐ through Q₁₀ₐ do not satisfy the condition, it is determined that the subject is “not in a fatigue state”.

From this perspective, in a preferred embodiment, a method of the present invention for evaluating fatigue includes the step of (1′) comparing a measured value of a glucose concentration in a biological sample obtained from a subject to a first threshold value, and further includes at least one of the steps of: (2′) comparing a measured value of a citrate concentration in the biological sample obtained from the subject with a second threshold value; (3′) comparing a measured value of a cis-aconitate concentration in the biological sample obtained from the subject with a third threshold value; (4′) comparing a measured value of an isocitrate concentration in the biological sample obtained from the subject with a fourth threshold value; (5′) comparing a measured value of a succinate concentration in the biological sample obtained from the subject with a fifth threshold value; (6′) comparing a measured value of a malate concentration in the biological sample obtained from the subject with a sixth threshold value; and (7′) comparing a measured value of a lactate concentration in the biological sample obtained from the subject with a seventh threshold value. The method of the present invention for evaluating fatigue preferably includes at least one of the steps (3′) through (5′), more preferably includes at least the step (5′), still more preferably includes the step (5′) and the step (4′) that are carried out in this order, further more preferably includes the step (5′), the step (4′), and the step (3′) that are carried out in this order. The method of the present invention for evaluating fatigue in accordance with the present embodiment may further include the step of assessing whether or not the measured values are smaller than the corresponding threshold values in the steps (1′) through (7′). This makes it possible to assess, with 95% or higher probability, whether or not a subject is suffering from chronic fatigue syndrome.

Further, an analysis such as a discriminant analysis, Partial Least Square, or Support Vector Machine with the use of at least one of the biomarkers Q₁₃ through Q₁₈ of Third Embodiment (or at least one of the biomarkers Q₁₃ₐ through Q₁₈ₐ) makes it possible to, without use of the standard values, evaluate (diagnose or assess) whether or not a subject is suffering from chronic fatigue syndrome. For example, a more sophisticated assessment can be achieved by subjecting any three (e.g., Q₁₃ through Q₁₅) of the biomarkers Q₁₃ through Q₁₈ to the analysis such as discriminant analysis, Partial Least Square, or Support Vector Machine. Especially a typical discriminant analysis is preferably carried out. In this case, a value is entered into all of M₁ through M₄ in the following formula (B), and it is determined, on the basis of a result thus obtained, whether or not a subject is suffering from chronic fatigue syndrome:

(a*M₂/M₁+b*M₃/M₂+c*M₄/M₃)+d (B)

Further, a higher-quality assessment can be achieved by subjecting any three (e.g., Q₁₃ₐ through Q₁₅ₐ) of the biomarkers Q₁₃ₐ through Q₁₈ₐ to the analysis such as discriminant analysis, Partial Least Square, or Support Vector Machine. Especially a typical discriminant analysis is preferably carried out. In this case, a value is entered to all of M₁ₐ, M₂ₐ, M₄ₐ, and M₅ₐ in the following formula (B′), and it is determined, on the basis of a result thus obtained, whether or not a subject is suffering from chronic fatigue syndrome:

(a*M₂ₐ/M₁ₐ+b*M₄ₐ/M₂ₐ+c*M₅ₐ/M₄ₐ)+d (B′)

From this perspective, in a preferred embodiment, a method of the present invention for evaluating fatigue includes at least three of the steps of: (1) obtaining a measured value of a glucose concentration in a biological sample obtained from a subject; (2) obtaining a measured value of a citrate concentration in the biological sample obtained from the subject; (3) obtaining a measured value of a cis-aconitate concentration in the biological sample obtained from the subject; (4) obtaining a measured value of an isocitrate concentration in the biological sample obtained from the subject; and (5) obtaining a measured value of a succinate concentration in the biological sample obtained from the subject, and preferably further includes the step of analyzing the measured values thus obtained.

The method of the present invention for evaluating fatigue has been thus described in accordance with First and Second Embodiments of biomarkers of the present invention. However, the method of the present invention for evaluating fatigue is not limited to those described above, and a person skilled in the art who read the present specification would easily understand that the scope of the method of the present invention for evaluating fatigue also encompasses, for example, (A) a method for evaluating fatigue, without the use of standard values, by using a measured value of a glucose concentration, a measured value of a citrate concentration, and a measured value of a cis-aconitate concentration (optionally using a measured value of an isocitrate concentration, a measured value of a malate concentration, and a measured value of a lactate concentration) in a biological sample obtained from a subject and (B) a method for evaluating fatigue by comparing measured values in a biological sample obtained from a subject with respective standard values.

The method of the present invention for evaluating fatigue may further include the step of measuring a glucose concentration, a citrate concentration, and a cis-aconitate concentration in the biological sample obtained from the subject and may optionally further include the step of measuring an isocitrate concentration, a malate concentration, and a lactate concentration. That is, the method of the present invention for evaluating fatigue may be executed on the basis of a glucose concentration, a citrate concentration, and a cis-aconitate concentration (and optionally on the basis of an isocitrate concentration, a malate concentration, and a lactate concentration) each measured in advance, or may start from a stage of measuring a glucose concentration, a citrate concentration, and a cis-aconitate concentration (and optionally an isocitrate concentration, a malate concentration, and a lactate concentration) in a biological sample obtained from a subject.

In the method of the present invention for evaluating fatigue, the first through third standard values are preferably an average of glucose concentrations, an average of citrate concentrations, and an average of cis-aconitate concentrations in respective biological samples obtained from a plurality of healthy subjects, respectively, but are not limited to such average values, and can be modes obtained by binominal distribution, or the like. Similarly, the fourth through seventh reference values are preferably an average of isocitrate concentrations, an average of succinate concentrations, an average of malate concentrations, and an average of lactate concentration in respective biological samples obtained from a plurality of healthy subjects, respectively, but are not limited to such average values, and can be modes obtained binominal distribution, or the like. Further, these values may be predefined values or may be values calculated on the basis of concentrations of metabolites in biological samples that are obtained from healthy subjects at the same time as sampling from a subject.

In order to execute the present invention, it is necessary to measure concentrations of metabolites (a glucose concentration, a citrate concentration, a cis-aconitate concentration, an isocitrate concentration, a succinate concentration, a malate concentration, and a lactate concentration) in a biological sample. An enzyme reaction using, as a substrate, glucose, citrate, cis-aconitate, isocitrate, succinate, malate, or lactate is well known in the art. This is clear from the fact that measurement kits using such techniques are commercially available. That is, a person skilled in the art can successfully measure concentrations of metabolites in a biological sample by optionally using any of such various techniques.

[3-2] Kit for Evaluating Fatigue

A kit including a reagent for use in the method for evaluating fatigue is also encompassed within the scope of the present invention. That is, in order to evaluate fatigue, a first kit of the present invention includes a fifth reagent for measuring a succinate concentration. As needed, the first kit of the present invention may further include a fourth reagent for measuring an isocitrate concentration, and further include at least one reagent selected from the group consisting of a first reagent for measuring a glucose concentration, a second reagent for measuring a citrate concentration, a third reagent for measuring a cis-aconitate concentration, a sixth reagent for measuring a malate concentration, and a seventh reagent for measuring a lactate concentration.

The first kit preferably further includes an instruction describing a standard value of a succinate concentration. As needed, the instruction may further describe a standard value of an isocitrate concentration, further describe a standard value of a cis-aconitate concentration, and further describe standard values of a glucose concentration, a citrate concentration, a malate concentration, and a lactate concentration.

An enzyme reaction whose substrate is, glucose, citrate, cis-aconitate, isocitrate, succinate, malate, or lactate is well known in the art. That is, a person skilled in the art can successfully measure concentrations of metabolites in a biological sample by optionally using any of such various techniques. For example, the first reagent may be Glucose oxidase or peroxidase each of which is an enzyme whose substrate is glucose. In this case, it is only necessary that the kit of the present invention include, as a suitable coloring reagent, a reagent (e.g., o-Dianisidine) which causes coloring by a redox reaction. The second reagent may be Citrate Lyase or Malic dehydrogenase each of which is an enzyme whose substrate is citrate. In this case, the kit of the present invention preferably includes, as a suitable coloring reagent, β-NADH. The third reagent may be Aconitase or Citrate Lyase each of which is an enzyme whose substrate is cis-aconitate. In this case, the kit of the present invention preferably includes, as a suitable coloring reagent, β-NADH or phenylhydrazine. The fourth reagent may be Isocitrate Lyase which is an enzyme whose substrate is isocitrate. In this case, the kit of the present invention preferably includes, as a suitable coloring reagent, phenylhydrazine. The fifth reagent may be Succinyl CoA Synthetase, Pyruvate Kinase, or Lactate Dehydrogenase, each of which is an enzyme whose substrate is succinate. In this case, the kit of the present invention preferably includes, as a suitable coloring reagent, β-NADH. The sixth reagent may be Malate Dehydrogenase which is an enzyme whose substrate is malate. In this case, the kit of the present invention preferably includes, as a suitable coloring reagent, NAD+. The seventh reagent may be Lactate Dehydrogenase or Glutamic Pyruvic Transaminase, each of which is an enzyme whose substrate is lactate. In this case, the kit of the present invention preferably includes, as a suitable coloring reagent, NAD+.

Further, a kit that makes it possible to visually detect the biomarkers Q₁ through Q₇ obtained by the method for evaluating fatigue is also encompassed within the scope of the present invention. That is, in order to evaluate fatigue, a second kit of the present invention includes a fifth presentation section which presents a fifth ratio, may further include a fourth presentation section which presents a fourth ratio, and may further include at least one presentation section selected from the group consisting of a first presentation section which presents a first ratio, a second presentation section which presents a second ratio, a third presentation section which presents a third ratio, a sixth presentation section which presents a sixth ratio, and a seventh presentation section which presents a seventh ratio. The second kit of the present invention may include separate members in which the first through seventh presentation sections are respectively provided or may include a single member in which all of the first through seventh presentation sections are provided.

Each of the first through seventh presentation sections in the second kit is preferably configured to present a visible hue corresponding to a measured value, and the second kit preferably includes an instruction describing references to be compared with the colors thus presented. The first presentation section presents a first color C₁ on the basis of R₁, the second presentation section presents a second color C₂ on the basis of R₂, and the biomarker Q₁ (i.e., a value based on a difference between C₁ and C₂) can be obtained by comparing C₁, C₂, and the references. The third presentation section presents a third color C₃ on the basis of R₃, and the biomarker Q₂ (i.e., a value based on a difference between C₂ and C₃) can be obtained by comparing C₂, C₃, and the references. A fatigue status of a subject can be thus evaluated with the use of the biomarkers (Q₁ and Q₂) obtained by the second kit of the present invention. As needed, the fourth presentation section presents a fourth color C₄ on the basis of R₄, and the biomarker Q₃ (i.e., a value based on a difference between C₃ and C₄) can be obtained by comparing C₃, C₄, and the references. It is therefore possible to use the biomarker Q₃ along with the biomarker Q₁ and the biomarker Q₂. A person skilled in the art who read the present specification could provide and use the fifth through seventh presentation sections in a similar manner. For the first through seventh presentation sections, chromogenic enzyme assay which utilize enzymes whose substrates are glucose, citrate, cis-aconitate, isocitrate, succinate, malate, and lactate can be used respectively.

The term “kit” used herein refers to a package including a container (e.g., a bottle, a plate, a tube, a dish, or the like) for containing a specific material therein, but a form in which a material is contained in a substance which is a composition is also encompassed by the term “kit”. The kit preferably includes a written direction for using the materials. The term “include(s) (including)” used herein in the aspect of a kit refers to a state of being contained in any one of containers constituting the kit. The kit of the present invention can be a package in which a plurality of different compositions are contained. In a case where the plurality of different compositions are in a liquid state, the plurality of different compositions may be contained in a container of the kit of the present invention. The kit of the present invention may contain the plurality of different compositions in a single container in a mixed manner or may contain the plurality of different compositions in respective separate containers. The “instruction” may be written or printed on a sheet of paper or another medium or may be stored in an electronic medium such as a magnetic tape, a computer-readable disc or tape, or a CD-ROM. The kit of the present invention can include a container containing a diluent, a solvent, a cleaning liquid, or other reagents. The kit of the present invention may include an appliance and a reagent that are necessary to obtain a biological sample. The kit of the present invention may include an appliance and a reagent that are necessary to prepare a target substance from a biological sample.

With the arrangements, the kit of the present invention can be easily used in an actual medical practice. This makes it possible to provide a speedier, less expensive, and objective diagnosis for fatigue.

Use of the kit of the present invention makes it possible to not only evaluate fatigue and propose a treatment of fatigue, but also provide a judgment criterion for fatigue (including chronic fatigue and chronic fatigue syndrome). This makes it easy to make a diagnosis as to whether or not a subject is in a fatigue state. That is, the kit of the present invention can be a kit for providing a diagnostic criterion or a judgment criterion for fatigue (including chronic fatigue and chronic fatigue syndrome) (e.g., a kit for obtaining data for diagnosing or assessing fatigue (including chronic fatigue and chronic fatigue syndrome)).

[3-3] Fatigue Evaluating System

A system used to execute the method for evaluating fatigue is also encompassed within the scope of the present invention. The embodiments below deal with an example in which members constituting a system of the present invention for evaluating fatigue are each “a functional block realized by causing computing means such as a CPU to execute a program code stored in a recording medium such as a ROM or a RAM”. Note, however, that each of the members constituting a system of the present invention for evaluating fatigue may be realized by hardware carrying out similar processing or may be realized by a combination of hardware carrying out a part of processing and the computing means executing a program code for controlling the hardware and carrying out remaining processing. Further, even a member explained as hardware out of the aforementioned members may be realized by a combination of hardware carrying out a part of processing and the computing means executing a program code for controlling the hardware and carrying out remaining processing. The number of computing means is not limited in particular. Single computing means may be provided, or a plurality of computing means connected via buses or various communication paths within a device may corporate to execute a program code.

The system of the present invention for evaluating fatigue includes, as its functional blocks, a measuring section 11, a storage section 12, a CPU 13, and a display section 14. The measuring section 11 has functions as measuring sections 11a through 11g for measuring a glucose concentration, a citrate concentration, a cis-aconitate concentration, an isocitrate concentration, a succinate concentration, a malate concentration, and a lactate concentration, respectively. The storage section 12 has functions as (A) a measured value receiving section 12a for receiving measured values M₁ through M₇ of a glucose concentration, a citrate concentration, a cis-aconitate concentration, an isocitrate concentration, a succinate concentration, a malate concentration, and a lactate concentration, and (B) a standard value storage section 12b in which standard values B₁ through B₇ for the glucose concentration, the citrate concentration, the cis-aconitate concentration, the isocitrate concentration, the succinate concentration, the malate concentration, and the lactate concentration are stored. The CPU 13 has functions as (A) a computing section 13a for generating information for evaluation of fatigue and (B) an evaluating section 13b for receiving the information from the computing section 13a and evaluating fatigue. The display section 14 has a function as an evaluation result display section 14 for displaying a result of the evaluation made by the evaluating section 13b. Note that each of the functional blocks is realized by causing the CPU 13 to execute a program stored in the storage section 12 and control a peripheral circuit such as an input/output circuit (not illustrated).

In a system for evaluating fatigue in accordance with First Embodiment, the following Steps 11 through 16 concerning glucose are executed. The measuring section 11a measures a glucose concentration in a sample so as to obtain a glucose concentration measured value M₁ (S11). The measuring section 11a supplies the glucose concentration measured value M₁ thus obtained to the measured value receiving section 12a (S12). The computing section 13a reads out the glucose concentration measured value M₁ stored in the measured value receiving section 12a (S13). The computing section 13a reads out a glucose concentration standard value (first standard value) B₁ stored in the standard value storage section 12b (S14). The computing section 13a calculates a ratio (first ratio) R₁ of M₁ to B₁ (S15). The computing section 13a supplies the first ratio R₁ to the evaluating section 13b (S16).

In the system for evaluating fatigue in accordance with First Embodiment, Steps 21 through 26 concerning citrate that correspond to Steps 11 through 16, respectively are executed. As a result, the computing section 13a supplies a second ratio R₂ to the evaluating section 13b. Similarly, Steps 31 through 36 concerning cis-aconitate that correspond to Steps 11 through 16, respectively are executed. As a result, the computing section 13a supplies a third ratio R₃ to the evaluating section 13b. Similarly, Steps concerning isocitrate that correspond to Steps 11 through 16, respectively are executed. As a result, the computing section 13a supplies a fourth ratio R₄ to the evaluating section 13b.

Subsequently, the evaluating section 13b receives the first through fourth ratios R₁ through R₄ supplied from the computing section 13a, and compares the first ratio R₁ and the second ratio R₂ (S51). In a case where it is determined that the first ratio R₁ is larger than the second ratio R₂ (R₁>R₂), the evaluating section 13b determines that a subject is “in a fatigue state”, and supplies a result thus obtained to the evaluation result display section 14 (S52). In a case where it is determined that the first ratio R₁ is smaller than the second ratio R₂ (R₁<R₂), the evaluating section 13b compares the second ratio R₂ and the third ratio R₃ (S53). In a case where it is determined that the second ratio R₂ is larger than the third ratio R₃ (R₂>R₃), the evaluating section 13b determines that the subject is “in a fatigue state”, and supplies a result thus obtained to the evaluation result display section 14 (S54). In a case where it is determined that the second ratio R₂ is smaller than the third ratio R₃ (R₂<R₃), the evaluating section 13b compares the third ratio R₃ and the fourth ratio R₄ (S55). In a case where it is determined that the fourth ratio R₄ is larger than the third ratio R₃ (R₄>R₃), the evaluating section 13b determines that the subject is “in a fatigue state”, and supplies a result thus obtained to the evaluation result display section 14 (S56). In a case where it is determined that the fourth ratio R₄ is smaller than the third ratio R₃ (R₄<R₃), the evaluating section 13b determines that the subject is “not in a fatigue state”, and supplies a result thus obtained to the evaluation result display section 14 (S57).

In First Embodiment, Steps 41 through 46 need not necessarily be executed. That is, R₄ need not necessarily be used for evaluation of fatigue. In this case, after Step 54, in a case where it is determined that the second ratio R₂ is smaller than the third ratio R₃ (R₂<R₃), the evaluating section 13b determines that the subject is “not in a fatigue state”, and supplies a result thus obtained to the evaluation result display section 14 (S58).

Although the above description has dealt with an example in which the comparison between R₁ and R₂ is given priority in Steps 51 through 58, the comparison between R₂ and R₃ or the comparison between R₃ and R₄ may be given priority. Although the above description has dealt with an example in which all of R₁ through R₄ are obtained by concurrent execution of the four step groups (i.e., Steps 11 through 16, Steps 21 through 26, Steps 31 through 36, Steps 41 through 46), R₁ through R₄ need not necessarily be obtained concurrently. Although the present embodiment has been described by using an example in which a glucose concentration, a citrate concentration, a cis-aconitate concentration, and an isocitrate concentration are used, similar processes are carried out also in a case where a succinate concentration, a malate concentration, and a lactate concentration are used.

In the system of the present invention for evaluating fatigue, the following processes may be given priority over evaluation of fatigue based on differences among R₁ through R₇ (Second Embodiment). In Second Embodiment, the storage section 12 has functions as (A) the measured value receiving section 12a, (B) the standard value storage section 12b, and (C) a reference value storage section 12c in which reference values t₁ through t₇ for a glucose concentration, a citrate concentration, a cis-aconitate concentration, an isocitrate concentration, a succinate concentration, a malate concentration, and a lactate concentration are stored. The computing section 13a supplies a first ratio R₁ to the evaluating section 13b (S16), reads out the reference value (first reference value) t₁ for the glucose concentration from the reference value storage section 12c, and supplies the first reference value t₁ to the evaluating section 13b (S116). The evaluating section 13b receives and compares the first ratio R₁ and the first reference value t₁ (S150). In a case where it is determined that the first ratio R₁ is almost equal to the first reference value t₁ (R₁≈t₁), the evaluating section 13b then receives and compares the fifth ratio R₅ and the fifth reference value t₅ (S151). In a case where it is determined that the fifth ratio R₅ is smaller than the fifth reference value t₅ (R₅<t₅), the evaluating section 13b determines that a subject is “in a fatigue state”, and supplies a result thus obtained to the evaluation result display section 14 (S152). In a case where it is determined that the fifth ratio R₅ is larger than the fifth reference value t₅ (R₅≧t₅), the evaluating section 13b then receives and compares the fourth ratio R₄ and the fourth reference value t₄ (S153). In a case where it is determined that the fourth ratio R₄ is smaller than the fourth reference value t₄ (R₄<t₄), the evaluating section 13b determines that a subject is “in a fatigue state”, and supplies a result thus obtained to the evaluation result display section 14 (S154). In a case where it is determined that the fourth ratio R₄ is larger than the fourth reference value t₄ (R₄≧t₄), the evaluating section 13b then receives and compares the third ratio R₃ and the third reference value t₃ (S155). In a case where it is determined that the third ratio R₃ is smaller than the third reference value t₃ (R₃<t₃), the evaluating section 13b determines that a subject is “in a fatigue state”, and supplies a result thus obtained to the evaluation result display section 14 (S156). In a case where it is determined that the third ratio R₃ is larger than the third reference value t₃ (R₃≧t₃), the evaluating section 13b determines that a subject is “not in a fatigue state”, and supplies a result thus obtained to the evaluation result display section 14 (S157). Although the present embodiment has been described by using an example in which a succinate concentration, an isocitrate concentration, and a cis-aconitate concentration are used, similar processes are carried out also in a case where a glucose concentration, a citrate concentration, a malate concentration, and a lactate concentration are additionally used. Needless to say, fatigue can be evaluated on the basis of only a cis-aconitate concentration, an isocitrate concentration, and/or a succinate concentration each of which makes it possible to evaluate fatigue with high probability.

In the system of the present invention for evaluating fatigue, the following processes may be given priority. In the present embodiment, the storage section 12 has functions as (A) the measured value receiving section 12a, (B) the standard value storage section 12b, and (C) a threshold value storage section 12d in which threshold values T₁ through T₇ for a glucose concentration, a citrate concentration, a cis-aconitate concentration, an isocitrate concentration, a succinate concentration, a malate concentration, and a lactate concentration are stored. The computing section 13a supplies a fifth measured value M₅ to the evaluating section 13b, reads out a threshold value (fifth threshold value) T₅ for a succinate concentration from the threshold value storage section 12d, and supplies the fifth threshold value T₅ to the evaluating section 13b. The evaluating section 13b receives and compares the fifth measured value M₅ and the fifth threshold value T₅. In a case where it is determined that the fifth measured value M₅ is smaller than the fifth threshold value T₅ (M₅<T₅), the evaluating section 13b determines that a subject is “in a fatigue state”, and supplies a result thus obtained to the evaluation result display section 14. In a case where it is determined that the fifth measured value M₅ is larger than the fifth threshold value T₅ (M₅≧T₅), the evaluating section 13b then receives and compares the fourth measured value M₄ and the fourth threshold value T₄. In a case where it is determined that the fourth measured value M₄ is smaller than the fourth threshold value T₄ (M₄<T₄), the evaluating section 13b determines that the subject is “in a fatigue state”, and supplies a result thus obtained to the evaluation result display section 14. In a case where it is determined that the fourth measured value M₄ is larger than the fourth threshold value T₄ (M₄≧T₄), the evaluating section 13b then receives and compares the third measured value M₃ and the third threshold value T₃. In a case where it is determined that the third measured value M₃ is smaller than the third threshold value T₃ (M₃<T₃), the evaluating section 13b determines that the subject is “in a fatigue state”, and supplies a result thus obtained to the evaluation result display section 14. In a case where it is determined that the third measured value M₃ is larger than the third threshold value T₃ (M₃≧T₃), the evaluating section 13b determines that the subject is “not in a fatigue state”, and supplies a result thus obtained to the evaluation result display section 14.

Even in a case where the standard value storage section 12b is not present or even in a case where the first through seventh standard values B₁ through B₇ are not stored in the standard value storage section 12b, the system of the present invention for evaluating fatigue can evaluate whether a subject is in a fatigue state or not (Third Embodiment). The computing section 13a reads out the measured values M₁ through M₇ stored in the measured value receiving section 12a and supplies the measured values M₁ through M₇ to the evaluating section 13b. The computing section 13a reads out the measured values M₁ through M₇ stored in the measured value receiving section 12a and supplies the measured values M₁ through M₇ to the evaluating section 13b. Subsequently, the evaluating section 13b receives the first through seventh measured values M₁ through M₇ supplied by the computing section 13a, and then compares M₁ through M₇ (S61). For example, the evaluating section 13b supplies again, to the computing section 13a, values of M₂/M₁, M₃/M₂, M₄/M₃, M₃/M₁, M₄/M₁, and M₄/M₂ obtained from M₁ through M₄ (S62). The computing section 13a executes an analysis (e.g., discriminant analysis, Partial Least Square, Support Vector Machine) using, for example, the values of M₂/M₁, M₃/M₂, and M₄/M₃ thus supplied (S63). Upon receipt of a result of the analysis from the computing section 13a, the evaluating section 13b determines, on the basis of a cutoff value, whether or not a subject is suffering from chronic fatigue syndrome, and then supplies a result thus obtained to the evaluation result display section 14 (S64). Although the present embodiment has been described by using an example in which a glucose concentration, a citrate concentration, a cis-aconitate concentration, and an isocitrate concentration are used, similar processes are carried out also in a case where a succinate concentration, a malate concentration, and a lactate concentration are additionally used.

Although the present invention has been described by use of an arrangement in which (A) the measuring section 11 measures a glucose concentration, a citrate concentration, a cis-aconitate concentration, an isocitrate concentration, a succinate concentration, a malate concentration, and a lactate concentration and (B) such values obtained by the measuring section 11 are supplied to the measured value receiving section 12a, it is also possible to employ an arrangement in which a glucose concentration, a citrate concentration, a cis-aconitate concentration, an isocitrate concentration, a succinate concentration, a malate concentration, and a lactate concentration that are obtained in advance are directly supplied to the measured value receiving section 12a. In this case, the system of the present invention for evaluating fatigue need not necessarily include the measuring section 11.

Use of the system of the present invention makes it possible to not only evaluate fatigue and propose a treatment for fatigue, but also provide a judgment standard for fatigue (including chronic fatigue and chronic fatigue syndrome). This makes it easy to make a diagnosis as to whether or not a subject is in a fatigue state. That is, the system of the present invention can be a system for providing a diagnostic standard or a judgment standard for fatigue (including chronic fatigue and chronic fatigue syndrome) (e.g., a system for obtaining data for diagnosing or assessing whether or not a subject is suffering from fatigue (including chronic fatigue and chronic fatigue syndrome)).

The present invention is not limited to the description of the embodiments above, but may be altered by a skilled person within the scope of the claims. An embodiment based on a proper combination of technical means disclosed in different embodiments is encompassed in the technical scope of the present invention.

### EXAMPLES

Twenty healthy subjects (male-female ratio=1:1, average age=36.10; provided by Osaka City University Hospital) and twenty subjects with chronic fatigue syndrome (male-female ratio=1:1, average age=36.15; provided by Osaka City University Hospital) were surveyed. A capillary electrophoresis-mass spectrometer was used to measure a metabolite in blood plasma. Glucose analysis kit II (Biovision) was used to measure a blood sugar level. A statistical analysis was carried out by using a significance test, a correlation test, and path analysis using SPSS 17.0 and Amos 18.0.

Blood plasma samples were prepared from blood taken from the healthy subjects and the patients with chronic fatigue syndrome (CFS). The Institute for Advanced Biosciences, Keio University was commissioned to measure metabolites in the blood plasma samples that have been pretreated. As a result, although no quantitative difference in most of metabolites having a branched-chain amino acid was found between the healthy subjects and the patients with CFS, a significant decline in amounts of some of the metabolites was found in the patients with CFS. In FIG. 1, metabolites, forming a glycolytic system and a citric acid cycle, for which a quantitative difference was observed between the healthy subjects and the patients with CFS are presented.

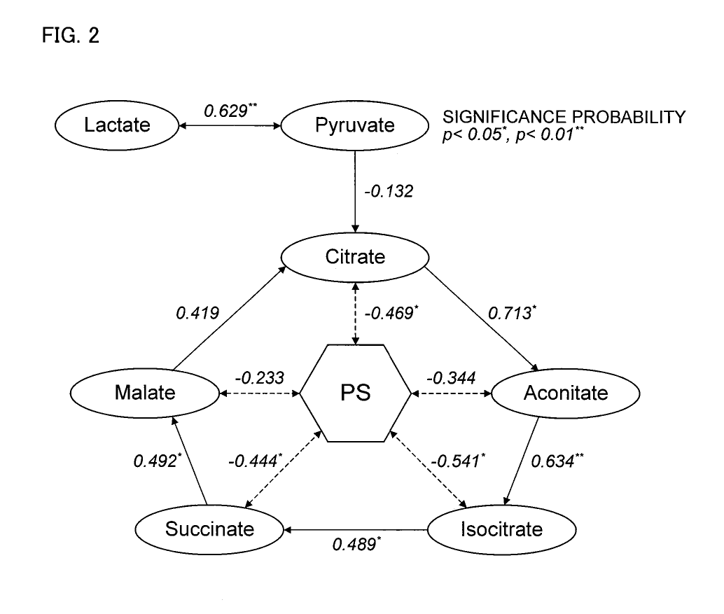

Next, correlations between the metabolites whose amounts in blood plasma significantly changed depending on presence/absence of fatigue and a performance status (PS) were examined. PS is a measure of a subjective severity of fatigue. A higher value of PS indicates a higher severity. The result is shown in FIG. 2. It was revealed that some of the examined metabolites showed a significant correlation with PS.

Further, a detailed analysis using a path analysis for estimating a causal relationship was carried out with respect to the metabolites whose amounts in blood plasma significantly changed depending on presence/absence of fatigue and which showed a significant correlation with PS. Since it brings to mind, based on the results obtained so far, that a glycolytic system and a citric acid cycle of the patients with CFS had abnormality, blood sugar levels in identical samples were measured independently and similarly subjected to the path analysis. As a result, it was revealed that the patients with CFS could be distinguished from the healthy subjects on the basis of amounts of three metabolites in blood plasma (glucose, citrate, and cis-aconitate) out of the examined metabolites (FIG. 3). FIG. 3 shows a comparison between the healthy subjects and the patients with CFS in terms of the metabolites. In FIG. 3, an average value of the healthy subjects is set to 100. It was also revealed that additional use of an amount of another metabolite (isocitrate) in blood plasma makes it possible to more easily distinguish the patients with CFS from the healthy subjects (FIG. 3).

As a result of an analysis based on a discriminant analysis in which a citrate/glucose ratio, a cis-aconitate/citrate ratio, and an isocitrate/cis-aconitate ratio that were calculated on the basis of measured values of these metabolites were concurrently evaluated on three axes, the normal group and the patients with chronic fatigue syndrome could be almost completely distinguished from each other (FIG. 4). Since a gradient of isocitrate/cis-aconitate is gradual as compared with a gradient of citrate/glucose and a gradient of cis-aconitate/citrate in FIG. 3, it was considered that isocitrate may be less important for distinguishing between the normal group and the patients with chronic fatigue syndrome than the other three metabolites. However, the result shown in FIG. 4 was beyond such estimation. Note that it was revealed that the patients with CFS could be distinguished with 90% or higher accuracy not only by the analysis based on a discriminant analysis, but also by an analysis based on Partial Least Square, Support Vector Machine, or the like (data is not shown).

Other metabolites were examined, too. As a result, it was revealed that an amount of succinate in blood plasma is also useful for distinguishing between the patients with CFS and the healthy subjects (FIG. 5). Although cis-aconitate is unstable and its concentration in a sample is not easy to measure, use of a succinate concentration in a sample makes it possible to more easily carry out the present invention. As a result of an analysis based on a discriminant analysis in which a citrate/glucose ratio, an isocitrate/citrate ratio, and a succinate/isocitrate ratio were concurrently evaluated on three axes, the normal group and the patients with chronic fatigue syndrome could be almost completely distinguished from each other (FIG. 6). Note that it was revealed that the patients with CFS could be distinguished with 90% or higher accuracy not only by the analysis based on a discriminant analysis, but also by an analysis based on Partial Least Square, Support Vector Machine, or the like (data is not shown).

The measured values of the metabolites in the blood plasma samples were inputted to analysis software R (free software, version 2.11.1) and a Random Forest program was executed. Random Forest is an algorism used for discrimination, regression, and clustering. As shown in FIG. 7, it was revealed that (A) out of a plurality of measurement items, isocitrate, succinate, and cis-aconitate are very important for distinguishing between the healthy subjects and patients with chronic fatigue and (B) it can be determined whether or not a subject is suffering from chronic fatigue syndrome, by measuring any one of isocitrate, succinate, and cis-aconitate.

The measured values of the metabolites (the compounds shown in FIG. 7) in the blood plasma samples were inputted to the analysis software R and a tree-based model program was executed. This program is used for planning and attainment of a goal in the field of decision theory. As shown in Table 1, it was revealed that (A) out of a plurality of measurement items, succinate is the most important factor for distinguishing between the healthy subjects and patients with chronic fatigue and (B) it can be determined with 95% or higher probability that a subject is suffering from chronic fatigue syndrome, in a case where a succinate measured value is lower than a threshold value T2 (14.56 μM). Next, the remaining measured values except for the succinate measured value were inputted to the analysis software R and the tree-based model program was executed. As a result, as shown in Table 1, it was revealed that (A) out of the plurality of measurement items except for succinate, isocitrate is the most important factor for distinguishing between the healthy subjects and patients with chronic fatigue and (B) it can be determined with 95% or higher probability that a subject is suffering from chronic fatigue syndrome, in a case where an isocitrate measured value is lower than a threshold value T1 (7.46 μM). Further, the remaining measured values except for the succinate measured value and the isocitrate measured value were inputted to the analysis software R and the tree-based model program was executed.

As a result, as shown in Table 1, it was revealed that (A) out of the plurality of measurement items except for succinate and isocitrate, cis-aconitate is the most important factor for distinguishing between the healthy subjects and patients with chronic fatigue and (B) it can be determined with 95% probability that a subject is suffering from chronic fatigue syndrome, in a case where a cis-aconitate measured value is lower than a threshold value T3 (10.66 μM).

In a case where the remaining measured values except for the succinate measured value, the isocitrate measured value, and the cis-aconitate measured value were inputted to the analysis software R and the tree-based model program was executed, a probability of determining that a subject is suffering from chronic fatigue syndrome was lower than 80%. In a case where malate is measured together with glutamic acid and isoleucine, the probability of determining that a subject is suffering from chronic fatigue syndrome becomes 95% or higher, and in a case where lactate is measured together with citrate and creatine, the probability of determining that a subject is suffering from chronic fatigue syndrome becomes 95% or higher. However, this is not very preferable since it becomes necessary to measure amounts of a plurality of substances in blood plasma.

As described above, it was revealed that it could be determined with 95% probability that a subject is suffering from chronic fatigue syndrome, by measuring any one of isocitrate, succinate, and cis-aconitate in blood plasma of the subject. Especially succinate is most important, isocitrate is second most important, and cis-aconitate is third most important. Accordingly, succinate is most preferably measured in a case where assessment of chronic fatigue syndrome is carried out with the use of a single metabolite. In a case where assessment of chronic fatigue syndrome is carried out with the use of a plurality of metabolites, measurement is most preferably carried out in the order of succinate and isocitrate or in the order of succinate, isocitrate, and cis-aconitate.

Note that there is a possibility that a calculated threshold value varies depending on which method for obtaining a measured value is used. In view of this possibility, it is preferable to determine that a subject is suffering from chronic fatigue syndrome, in a case where any one of t1, t2, and t3 is smaller than a minimum value. Note that t1 through t3 in the present Example represent ratios (%) of measured values of isocitrate, succinate, and cis-aconitate in a subject to average values of isocitrate, succinate, and cis-aconitate in the healthy subjects, respectively. A reference value is a value obtained as follows:

t=(threshold value T/average value of healthy subjects)×100%

In the analyzed compounds and analysis software used in the present Example, reference values for isocitrate, succinate, and cis-aconitate are 66.0%, 66.6%, and 69.3%, respectively.

TABLE 1

| | Condition for | | | |
|---|---|---|---|---|
| | determining that | | | |
| | subject is suffering | | | |
| | from CFS | | | |
| | Threshold | Reference | | Sensitivity |
| | value | value | Accuracy | Specificity |
| Isocitrate | <7.45 μM | <66.0% | 97.50% | 95% |
| | | | | 100% |
| Succinate | <14.55 μM | <66.6% | 97.50% | 95% |
| | | | | 100% |
| Cis- | <10.65 μM | <69.3% | 97.50% | 100% |
| Aconitate | | | | 95% |
| Malate | <13.37 μM | | 87.50% | 100% |
| | | | | 75% |
| Lactate | <2609.83 μM | | 77.50% | 75% |
| | | | | 80% |
| Citrate | <107.6 μM | | 77.50% | 60% |
| | | | | 95% |

Use of the present invention makes it possible to diagnose fatigue with the use of a simple kit. Further, an effective treatment may be found on the basis of a diagnosis result obtained by using the present invention. For example, application of the present invention to a patient who has a strong feeling of fatigue or a long-term fatigue makes it possible to objectively evaluate a degree of fatigue and to determine whether or not the patient is suffering from chronic fatigue syndrome. Evaluation of a degree of fatigue by use of the present invention does not require a high level of knowledge about fatigue, and therefore can be carried out also in general medical facilities. Further, use of the present invention makes it possible to estimate which part of an energy (ATP) production metabolic system of a living body has abnormality. This makes it possible to improve a lifestyle guidance for a patient and to provide food/drug which promotes a metabolic system that is considered as a cause or food/drug which promotes energy production even under abnormal metabolism.

The present invention is not limited to the description of the embodiments above, but may be altered by a skilled person within the scope of the claims. An embodiment based on a proper combination of technical means disclosed in different embodiments is encompassed in the technical scope of the present invention.

### INDUSTRIAL APPLICABILITY

Use of the present invention makes it possible to objectively and easily evaluate and diagnose fatigue. Further, the present invention can provide a technique useful for treatment of fatigue. Since fatigue is a very important health issue, realization of evaluation, diagnosis, and treatment of fatigue greatly contributes to every kind of industries.

1. A method for evaluating fatigue, comprising at least two steps selected from the group consisting of:
(a) obtaining a first ratio of a first measured value to a first standard value, the first measured value being a measured value of a glucose concentration in a biological sample obtained from a subject;
(b) obtaining a second ratio of a second measured value to a second standard value, the second measured value being a measured value of a citrate concentration in the biological sample obtained from the subject;
(c) obtaining a third ratio of a third measured value to a third standard value, the third measured value being a measured value of a cis-aconitate concentration in the biological sample obtained from the subject;
(d) obtaining a fourth ratio of a fourth measured value to a fourth standard value, the fourth measured value being a measured value of an isocitrate concentration in the biological sample obtained from the subject;
(e) obtaining a fifth ratio of a fifth measured value to a fifth standard value, the fifth measured value being a measured value of a succinate concentration in the biological sample obtained from the subject;
(f) obtaining a sixth ratio of a sixth measured value to a sixth standard value, the sixth measured value being a measured value of a malate concentration in the biological sample obtained from the subject; and
(g) obtaining a seventh ratio of a seventh measured value to a seventh standard value, the seventh measured value being a measured value of a lactate concentration in the biological sample obtained from the subject.

2. The method according to claim 1, further comprising the step of comparing at least one pair of ratios out of at least two ratios obtained by the at least two steps.

3. The method according to claim 2, comprising at least two steps selected from the group consisting of the step (a), the step (b), the step (c), the step (d), and the step (e).

4. The method according to claim 1, comprising (A) the step (a) and (B) at least one step selected from the group consisting of the steps (b) through (g).

5. The method according to claim 4, further comprising the step of comparing at least one ratio obtained by the at least one step with a threshold value corresponding to the at least one ratio.

6. The method according to claim 4, wherein the at least one step is at least one step selected from the group consisting of the steps (c) through (e).

7. The method according to claim 6, wherein the step (d) is carried out subsequently to the step (e).

8. The method according to claim 7, wherein the step (c) is carried out subsequently to the step (d).

9. The method according to claim 4, which is carried out when the first ratio is substantially 1.

10. A method for evaluating fatigue, comprising at least one step selected from the group consisting of:
(a) comparing a first measured value with a first threshold value, the first measured value being a measured value of a glucose concentration in a biological sample obtained from a subject;
(b) comparing a second measured value with a second threshold value, the second measured value being a measured value of a citrate concentration in the biological sample obtained from the subject;
(c) comparing a third measured value with a third threshold value, the third measured value being a measured value of a cis-aconitate concentration in the biological sample obtained from the subject;
(d) comparing a fourth measured value with a fourth threshold value, the fourth measured value being a measured value of an isocitrate concentration in the biological sample obtained from the subject;
(e) comparing a fifth measured value with a fifth threshold value, the fifth measured value being a measured value of a succinate concentration in the biological sample obtained from the subject;
(f) comparing a sixth measured value with a sixth threshold value, the sixth measured value being a measured value of a malate concentration in the biological sample obtained from the subject; and
(g) comparing a seventh measured value with a seventh threshold value, the seventh measured value being a measured value of a lactate concentration in the biological sample obtained from the subject.

11. The method according to claim 10, comprising at least one step selected from the group consisting of: at least one of the steps (c) through (e).

12. The method according to claim 11, wherein the step (d) is carried out subsequently to the step (e).

13. The method according to claim 12, wherein the step (c) is carried out subsequently to the step (e).

14. A method for evaluating fatigue, comprising the step of obtaining a ratio of at least one pair of measured values out of at least two measured values selected from the group consisting of a measured value of a glucose concentration, a measured value of a citrate concentration, a measured value of a cis-aconitate concentration, a measured value of an isocitrate concentration, and a measured value of a succinate concentration in a biological sample obtained from a subject.

15. The method according to claim 14, further comprising the step of analyzing the ratio of the at least one pair of measured values by a discriminant analysis, Partial Least Square, or Support Vector Machine.

16. A kit for evaluating fatigue, comprising a fifth reagent for measuring a succinate concentration.

17. The kit according to claim 16, further comprising a fourth reagent for measuring an isocitrate concentration.

18. The kit according to claim 17, further comprising at least one of a first reagent for measuring a glucose concentration, a second reagent for measuring a citrate concentration, a third reagent for measuring a cis-aconitate concentration, a sixth reagent for measuring a malate concentration, and a seventh reagent for measuring a lactate concentration.

19. A kit for evaluating fatigue, comprising a fifth presentation section which presents a fifth ratio of a measured value of a succinate concentration to a fifth standard value.

20. The kit according to claim 19, further comprising a fourth presentation section which presents a fourth ratio of a measured value of an isocitrate concentration to a fourth standard value.

21. The kit according to claim 20, further comprising at least one of a first presentation section which presents a first ratio of a measured value of a glucose concentration to a first standard value, a second presentation section which presents a second ratio of a measured value of a citrate concentration to a second standard value, a third presentation section which presents a third ratio of a measured value of a cis-aconitate concentration to a third standard value, a sixth presentation section which presents a sixth ratio of a measured value of a malate concentration to a sixth standard value, and a seventh presentation section which presents a seventh ratio of a measured value of a lactate concentration to a seventh standard value.

22. A system for evaluating fatigue, comprising:
a measured value receiving section which receives at least two measured values selected from the group consisting of a measured value of a glucose concentration, a measured value of a citrate concentration, a measured value of a cis-aconitate concentration, a measured value of an isocitrate concentration, a measured value of a succinate concentration, a measured value of a malate concentration, and a measured value of a lactate concentration;
a standard value storage section in which at least two standard values are stored, the at least two standard values being selected from the group consisting of first through seventh standard values that correspond to the glucose concentration, the citrate concentration, the cis-aconitate concentration, the isocitrate concentration, the succinate concentration, the malate concentration, and the lactate concentration, respectively;
a computing section which receives (A) a two measured value from the measured value receiving section and (B) a standard value from the standard value storage section and generates information for evaluating fatigue; and
an evaluating section which receives the information from the computing section and evaluates fatigue,
the computing section calculating at least two ratios selected from the group consisting of:
a first ratio of the measured value of the glucose concentration to the first standard value,
a second ratio of the measured value of the citrate concentration to the second standard value,
a third ratio of the measured value of the cis-aconitate concentration to the third standard value,
a fourth ratio of the measured value of the isocitrate concentration to the fourth standard value,
a fifth ratio of the measured value of the succinate concentration to the fifth standard value,
a sixth ratio of the measured value of the malate concentration to the sixth standard value, and
a seventh ratio of the measured value of the lactate concentration to the seventh standard value.

23. The system according to claim 22, wherein the computing section executes a comparison between at least one pair of ratios out of the at least two ratios.

24. The system according to claim 23, wherein:
the measured value receiving section receives at least two measured values selected from the group consisting of the measured value of the glucose concentration, the measured value of the citrate concentration, the measured value of the cis-aconitate concentration, the measured value of the isocitrate concentration, and the measured value of the succinate concentration,
the standard value storage section stores therein at least two standard values selected from the group consisting of the first through fifth standard values that correspond to the glucose concentration, the citrate concentration, the cis-aconitate concentration, the isocitrate concentration, and the succinate concentration, respectively, and
the computing section further calculates at least two ratios selected from the group consisting of the first through fifth ratios and executes a comparison between at least one pair of ratios out of the at least two ratios.

25. The system according to claim 24, wherein:
the computing section calculates (A) the first ratio and (B) at least one ratio selected from the group consisting of the second through seventh ratios.

26. The system according to claim 25, wherein:
the standard value storage section stores therein at least one threshold value selected from the group consisting of threshold values corresponding to the second through seventh ratios, and
the computing section receives the at least one threshold value from the standard value storage section and executes a comparison between the at least one ratio calculated by the computing section and the at least one threshold value corresponding to the at least one ratio.

27. The system according to claim 25, wherein the computing section calculates at least one of the third through fifth ratios.

28. The system according to claim 27, wherein the computing section calculates the fifth ratio subsequently to the fourth ratio.

29. The system according to claim 28, wherein the computing section calculates the third ratio subsequently to the fifth ratio.

30. The system according to claim 25, wherein an assessing section assesses whether or not the first ratio is substantially 1.

31. A system for evaluating fatigue, comprising:
a measured value receiving section which receives at least one measured value selected from the group consisting of a measured value of a glucose concentration, a measured value of a citrate concentration, a measured value of a cis-aconitate concentration, a measured value of an isocitrate concentration, a measured value of a succinate concentration, a measured value of a malate concentration, and a measured value of a lactate concentration;
a threshold value storage section in which at least one threshold value is stored, the at least one threshold value being selected from the group consisting of first through seventh threshold values that correspond to the glucose concentration, the citrate concentration, the cis-aconitate concentration, the isocitrate concentration, the succinate concentration, the malate concentration, and the lactate concentration, respectively;
a computing section which receives (A) a measured value from the measured value receiving section and (B) a threshold value from the threshold value storage section and generates information for evaluating fatigue; and
an evaluating section which receives the information from the computing section and evaluates fatigue,
the computing section executing at least one comparison selected from the group consisting of a first comparison between the measured value of the glucose concentration and the first threshold value, a second comparison between the measured value of the citrate concentration and the second threshold value, a third comparison between the measured value of the cis-aconitate concentration and the third threshold value, a fourth comparison between the measured value of the isocitrate concentration and the fourth threshold value, a fifth comparison between the measured value of the succinate concentration and the fifth threshold value, a sixth comparison between the measured value of the malate concentration and the sixth threshold value, and a seventh comparison between the measured value of the lactate concentration and the seventh threshold value.

32. The system according to claim 31, wherein the computing section executes at least one comparison selected from the group consisting of the third through fifth comparisons.

33. The system according to claim 32, wherein the fourth comparison is executed subsequently to the fifth comparison.

34. The system according to claim 33, wherein the third comparison is executed subsequently to the fifth comparison.

35. A system for evaluating fatigue, comprising:
a measured value receiving section which receives at least two measured values selected from the group consisting of a measured value of a glucose concentration, a measured value of a citrate concentration, a measured value of a cis-aconitate concentration, a measured value of an isocitrate concentration, and a measured value of a succinate concentration;
a computing section which receives a measured value from the measured value receiving section and generates information for evaluating fatigue; and
an evaluating section which receives the information from the computing section and evaluates fatigue,
the computing section executing a comparison between at least one pair of measured values out of the at least two measured values so as to obtain a ratio of the at least one pair of measured values.

36. The system according to claim 35, wherein the computing section executes a discriminant analysis, Partial Least Square, or Support Vector Machine on a basis of the ratio of the at least one pair of measured values.

37. The system according to claim 22, further comprising first through third measuring sections which measure the glucose concentration, the citrate concentration, and the cis-aconitate concentration, respectively, and optionally further comprising fourth through seventh measuring sections which measure the isocitrate concentration, the succinate concentration, the malate concentration, and the lactate concentration, respectively,
the measured value receiving section receiving values obtained by the first through seventh measuring sections.
